(19) United States
(12) Patent Application Publication
(10) Pub. No.: US 2022/0389372 A1
(43) Pub. Date: Dec. 8, 2022

(54) COMPOSITIONS AND METHODS FOR ENHANCED PROTEIN PRODUCTION IN BACILLUS CELLS

(71) Applicant: DANISCO US INC., Palo Alto, CA (US)

(72) Inventors: Cristina Bongiorni, Fremont, CA (US); Zhen Ma, Garnet Valley, PA (US); Anita Van Kimmenade, Woodside, CA (US)

(21) Appl. No.: 17/774,437

(22) PCT Filed: Nov. 10, 2020
(86) PCT No.: PCT/US2020/059830

§ 371 Date: May 4, 2022

Related U.S. Application Data
(60) Provisional application No. 62/933,536, filed on Nov. 11, 2019.

(51) Int. Cl. C12N 1/20; C12N 15/00; C12N 1/08; C07K 14/32

(52) U.S. Cl. CPC C12N 1/20; C12N 15/00; C12N 1/08; C07K 14/32

(57) ABSTRACT

The present disclosure is generally related to compositions and methods for constructing and obtaining Bacillus sp. host cells (strains) having enhanced protein production phenotypes. Certain embodiments of the disclosure are therefore related to genetically modified Bacillus sp. cells derived (obtained) from parental Bacillus sp. cells (e.g., such as parental Bacillus cells expressing/producing a protein of interest). Thus, certain embodiments are directed to modified Bacillus sp. cells expressing/producing an endogenous protein of interest, or a heterologous protein of interest, wherein the modified Bacillus sp. cell comprises a genetic modification of a yitMOP operon, a combined genetic modification of a yitMOP operon and a sdpABC operon, a genetic modification of a yitM gene and the like, wherein the modified Bacillus sp. cells produce an increased amount of the POI relative the parental cells from which they were derived (i.e., when grown/cultivated/fermented under identical conditions).

### FIELD

The present disclosure is generally related to the fields of bacteriology, microbiology, genetics, molecular biology, enzymology, industrial protein production the like. More particularly, the present disclosure is related to compositions and methods for obtaining Bacillus sp. cells having increased protein production capabilities.

### CROSS REFERENCE TO RELATED APPLICATIONS

This application claims benefit to U.S. Provisional Patent Application No. 62/933,536, filed Nov. 11, 2019, which is incorporated herein by referenced in its entirety.

### REFERENCE TO A SEQUENCE LISTING

The contents of the electronic submission of the text file Sequence Listing, named “NB41375-WO-PCT_SequenceListing.txt” was created on Oct. 23, 2020 and is 36 KB in size, which is hereby incorporated by reference in its entirety.

### BACKGROUND

Gram-positive bacteria such as Bacillus subtilis, Bacillus licheniformis and Bacillus amyloliquefaciens are frequently used as microbial factories for the production of industrial relevant proteins, due to their excellent fermentation properties and high yields (e.g., up to 25 grams per liter culture; Van Dijl and Hecker, 2013). For example, B. subtilis is well known for its production of α-amylases (Jensen et al., 2000; Raul et al., 2014) and proteases (Brode et al., 1996) necessary for food, textile, laundry, medical instrument cleaning, pharmaceutical industries and the like (Westers et al., 2004). Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority, and many of their products gained a “Generally Recognized As Safe” (GRAS) status from the US Food and Drug Administration (Olempska-Beer et al., 2006; Earl et al., 2008; Caspers et al., 2010).

Thus, the production of proteins (e.g., enzymes, antibodies, receptors, etc.) in microbial host cells is of particular interest in the biotechnological arts. Likewise, the optimization of Bacillus sp. host cells for the production and secretion of one or more protein(s) of interest is of high relevance, particularly in the industrial biotechnology setting, wherein small improvements in protein yield are quite significant when the protein is produced in large industrial quantities. Thus, the ability to modify and engineer Bacillus sp. host cells for enhanced protein production is highly desirable in the art. As described hereinafter, the instant disclosure addresses such needs in the art. More particularly, as presented, described and exemplified herein, certain embodiments of the disclosure are related to methods and compositions for constructing Bacillus sp. host cells capable of producing increased amounts of proteins of interest.

### SUMMARY

The present disclosure is generally related to compositions and methods for constructing Bacillus sp. cells having enhanced protein production phenotypes. Certain embodiments of the disclosure are related to genetically modified Bacillus sp. cells derived (obtained) from parental Bacillus sp. cells (e.g., such as parental Bacillus cells expressing/producing a protein of interest). For example, in certain embodiments, a modified Bacillus sp. cell of the disclosure is derived from a parental Bacillus sp. cell expressing/producing an endogenous protein of interest, or a heterologous protein of interest, wherein the modified Bacillus sp. cell comprises a genetic modification of a yitMOP operon and produces an increased amount of the POI relative the parental cell from which it was derived (i.e., when grown/cultivated/fermented under identical conditions). In certain other embodiments, a modified Bacillus sp. cell of the disclosure is derived (obtained) from a parental Bacillus sp. cell expressing/producing an endogenous protein of interest, or a heterologous protein of interest, wherein the modified Bacillus sp. cell comprises a combined genetic modification of the yitMOP operon and the sdpABC operon, wherein the modified Bacillus sp. cell produces an increased amount of the POI relative the parental cell (i.e., when grown/cultivated/fermented under identical conditions). In other embodiments, a modified Bacillus sp. cell of the disclosure is derived from a parental Bacillus sp. cell expressing/producing an endogenous protein of interest, or a heterologous protein of interest, wherein the modified Bacillus sp. cell comprises a genetic modification of the yitM gene, wherein the modified Bacillus sp. cell produces an increased amount of the POI relative the parental cell (i.e., when grown/cultivated/fermented under identical conditions). In yet other embodiments, a modified Bacillus sp. cell of the disclosure is derived from a parental Bacillus sp. cell expressing/producing an endogenous protein of interest, or a heterologous protein of interest, wherein the modified Bacillus sp. cell comprises a combined genetic modification of the yitM gene and the sdpABC operon, wherein the modified Bacillus sp. cell produces an increased amount of the POI relative the parental cell (i.e., when grown/cultivated/fermented under identical conditions).

### BRIEF DESCRIPTION OF THE DRAWINGS

FIG. 1 shows the nucleic acid sequences and encoded amino acid sequences of the native B. subtilis yitMOP operon. More specifically, FIG. 1A presents the nucleic acid sequence of the yitM open reading frame (ORF; SEQ ID NO: 1) encoding the yitM polypeptide (SEQ ID NO: 2) and the yitO open reading frame (ORF; SEQ ID NO: 3) encoding the yitO polypeptide (SEQ ID NO: 4), and FIG. 1B presents the nucleic acid sequence of the yitP open reading frame (ORF; SEQ ID NO: 5) encoding the yitP polypeptide (SEQ ID NO: 6).

FIG. 2 shows a beneficial effect on protein production during large scale fermentation of a modified B. subtilis strain (JL77) comprising a combined deletion of the yitMOP and sdpABC cannibalism operons (ΔyitMOP+ΔsdpABC). More particularly, as presented in FIG. 2, the cells of the JL77 strain and CB24-12 strain were tested in fermentors under identical standard conditions, wherein the average increase in V42 protease production at the end of fermentation for the JL77 strain was approximately a 10% increase relative to the amount of V42 protease produced by the CB24-12 strain at the end of fermentation.

### BRIEF DESCRIPTION OF THE BIOLOGICAL SEQUENCES

SEQ ID NO: 1 is a B. subtilis open reading frame (ORF) nucleic acid sequence encoding a native YitM polypeptide of SEQ ID NO: 2.

SEQ ID NO: 2 is the amino acid sequence of the B. subtilis YitM polypeptide encoded by SEQ ID NO: 1.

SEQ ID NO: 3 is a B. subtilis ORF sequence encoding a native YitO polypeptide of SEQ ID NO: 4.

SEQ ID NO: 4 is the amino acid sequence of the B. subtilis YitO polypeptide encoded by SEQ ID NO: 3.

SEQ ID NO: 5 is a B. subtilis ORF sequence encoding a native YitP polypeptide of SEQ ID NO: 6.

SEQ ID NO: 6 is the amino acid sequence of the B. subtilis YitP polypeptide encoded by SEQ ID NO: 5.

SEQ ID NO: 7 is the nucleic acid sequence of primer oJKL228.

SEQ ID NO: 8 is the nucleic acid sequence of primer oJKL229.

SEQ ID NO: 9 is the nucleic acid sequence of primer oJKL232.

SEQ ID NO: 10 is the nucleic acid sequence of primer oJKL223.

SEQ ID NO: 11 is the nucleic acid sequence of primer oJKL230.

SEQ ID NO: 12 is the nucleic acid sequence of primer oJKL231.

SEQ ID NO: 13 is the nucleic acid sequence of primer oJKL226.

SEQ ID NO: 14 is the nucleic acid sequence of primer oJKL227.

SEQ ID NO: 15 is the nucleic acid sequence of primer M13F.

SEQ ID NO: 16 is the nucleic acid sequence of primer M13R.

SEQ ID NO: 17 is the nucleic acid sequence of primer oJKL246.

SEQ ID NO: 18 is the nucleic acid sequence of primer 5′ spec out.

SEQ ID NO: 19 is the nucleic acid sequence of primer oJKL247.

SEQ ID NO: 20 is the nucleic acid sequence of primer 3′ spec out.

SEQ ID NO: 21 is the nucleic acid sequence of primer oJKL220.

SEQ ID NO: 22 is the nucleic acid sequence of primer oJKL221.

SEQ ID NO: 23 is the nucleic acid sequence of primer oJKL224.

SEQ ID NO: 24 is the nucleic acid sequence of primer oJKL225.

SEQ ID NO: 25 is the nucleic acid sequence of primer oJKL222.

SEQ ID NO: 26 is the nucleic acid sequence of primer oJKL223.

SEQ ID NO: 27 is the nucleic acid sequence of primer oJKL218.

SEQ ID NO: 28 is the nucleic acid sequence of primer oJKL2195.

SEQ ID NO: 29 is the nucleic acid sequence of primer oJKL238.

SEQ ID NO: 30 is the nucleic acid sequence of primer tet 3′ out.

SEQ ID NO: 31 is the nucleic acid sequence of primer oJKL229.

SEQ ID NO: 32 is the nucleic acid sequence of primer tet 5′ out.

SEQ ID NO: 33 is a Bacillus amyloliquefaciens open reading frame (ORF) nucleic acid sequence encoding a native YitP polypeptide of SEQ ID NO: 34.

SEQ ID NO: 34 is the amino acid sequence of the B. amyloliquefaciens YitP polypeptide encoded by SEQ ID NO: 33.

SEQ ID NO: 35 is a B. amyloliquefaciens ORF sequence encoding a native YitO polypeptide of SEQ ID NO: 36.

SEQ ID NO: 36 is the amino acid sequence of the B. amyloliquefaciens YitO polypeptide encoded by SEQ ID NO: 35.

SEQ ID NO: 37 is a B. amyloliquefaciens ORF sequence encoding a native YitM polypeptide of SEQ ID NO: 38.

SEQ ID NO: 38 is the amino acid sequence of the B. amyloliquefaciens YitM polypeptide encoded by SEQ ID NO: 37.

SEQ ID NO: 39 is a promoter region DNA sequence of the B. amyloliquefaciens yitMOP operon.

SEQ ID NO: 40 is a promoter region DNA sequence of the B. subtilis yitMOP operon.

SEQ ID NO: 41 is a promoter region DNA sequence of the B. subtilis sdpABC operon.

SEQ ID NO: 42 is a B. subtilis open reading frame (ORF) nucleic acid sequence encoding a native SdpA polypeptide of SEQ ID NO: 43.

SEQ ID NO: 43 is the amino acid sequence of the B. subtilis SdpA polypeptide encoded by SEQ ID NO: 42.

SEQ ID NO: 44 is a B. subtilis open reading frame (ORF) nucleic acid sequence encoding a native SdpB polypeptide of SEQ ID NO: 45.

SEQ ID NO: 45 is the amino acid sequence of the B. subtilis SdpB polypeptide encoded by SEQ ID NO: 44.

SEQ ID NO: 46 is a B. subtilis open reading frame (ORF) nucleic acid sequence encoding a native SdpC polypeptide of SEQ ID NO: 47.

SEQ ID NO: 47 is the amino acid sequence of the B. subtilis SdpC polypeptide encoded by SEQ ID NO: 46.

### DETAILED DESCRIPTION

The present disclosure is generally related to compositions and methods for constructing and obtaining Bacillus sp. cells having enhanced protein production phenotypes. Thus, certain embodiments of the disclosure are related to genetically modified Bacillus sp. cells derived (obtained) from parental Bacillus sp. cells (e.g., such as parental Bacillus cells expressing/producing a protein of interest). In certain embodiments, a parental Bacillus sp. cell comprises an endogenous gene encoding a protein of interest (POI). In certain embodiments, the endogenous gene encodes a protease or an amylase. In certain other embodiments, a parental Bacillus sp. cell comprises an expression cassette encoding a heterologous protein of interest (POI). For example, in certain embodiments, a modified Bacillus sp. cell of the disclosure is derived from a parental Bacillus sp. cell expressing/producing an endogenous protein of interest, or a heterologous protein of interest, wherein the modified Bacillus sp. cell comprises a genetic modification of a yitMOP operon and produces an increased amount of the POI relative the parental cell (i.e., when grown/cultivated/fermented under identical conditions). In certain other embodiments, a modified Bacillus sp. cell of the disclosure is derived from a parental Bacillus sp. cell expressing/producing an endogenous protein of interest, or a heterologous protein of interest, wherein the modified Bacillus sp. cell comprises a combined genetic modification of the yitMOP operon and the sdpABC operon, wherein the modified Bacillus sp. cell produces an increased amount of the POI relative the parental cell (i.e., when grown/cultivated/fermented under identical conditions). In yet other embodiments, a modified Bacillus sp. cell of the disclosure is derived from a parental Bacillus sp. cell expressing/producing an endogenous protein of interest, or a heterologous protein of interest, wherein the modified Bacillus sp. cell comprises a genetic modification of the yitM gene and produces an increased amount of the POI relative the parental cell (i.e., when grown/cultivated/fermented under identical conditions). In other embodiments, a modified Bacillus sp. cell of the disclosure is derived from a parental Bacillus sp. cell expressing/producing an endogenous protein of interest, or a heterologous protein of interest, wherein the modified Bacillus sp. cell comprises a combined genetic modification of the yitMOP operon (or the yitM gene) and the sdpABC operon, wherein the modified Bacillus sp. cell produces an increased amount of the POI relative the parental cell (i.e., when grown/cultivated/fermented under identical conditions).

### I. DEFINITIONS

In view of the modified Bacillus sp. cells of the disclosure and methods thereof described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described.

All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the phrase “host cell” refers to a cell that has the capacity to act as a host or expression vehicle for a newly introduced DNA sequence. Thus, in certain embodiments of the disclosure, host cells are for example, Bacillus sp. cells or E. coli cells.

As used herein, the terms “wild-type” and “native” are used interchangeably and refer to genes, promoters, proteins, protein mixes, cells or strains, as found in nature.

As used herein, the “genus Bacillus” or “Bacillus sp.” cells include all species within the genus “Bacillus” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

As used herein, “modified cells” refers to recombinant (host) cells that comprise at least one genetic modification which is not present in the “parental” host cell from which the modified cells are derived. For example, in certain embodiments, a “parental” cell is altered (e.g., via one or more genetic modifications introduced into the parental cell) to generate a “modified” (daughter) cell thereof. In certain embodiments, a parental cell may be referred to as a “control cell”, particularly when being compared with, or relative to, a “modified” Bacillus sp. (daughter) cell.

As used herein, when the expression and/or production of a protein of interest (POI) in an “unmodified” (parental) cell is being compared to the expression and/or production of the same POI in a “modified” (daughter) cell, it will be understood that the “modified” and “unmodified” cells are grown/cultivated/fermented under the same conditions (e.g., the same conditions such as media, temperature, pH and the like).

As used herein, a wild-type Bacillus subtilis “yitMOP operon” encodes a “YitM polypeptide”, a “YitO polypeptide” and a “YitP polypeptide”.

As used herein, a wild-type B. subtilis “yitM” nucleic acid sequence encodes a wild-type B. subtilis “YitM” polypeptide comprising SEQ ID NO: 2.

As used herein, a wild-type B. subtilis “yitO” nucleic acid sequence encodes a wild-type B. subtilis “YitO” polypeptide comprising SEQ ID NO: 4.

As used herein, a wild-type B. subtilis “yitP” nucleic acid sequence encodes a wild-type B. subtilis “YitP” polypeptide comprising SEQ ID NO: 6.

As used herein, a wild-type Bacillus amyloliquefaciens “yitMOP operon” encodes a “YitM polypeptide”, a “YitO polypeptide” and a “YitP” polypeptide”.

As used herein, a wild-type B. amyloliquefaciens “yitM” nucleic acid sequence encodes a wild-type B. amyloliquefaciens “YitM” polypeptide comprising SEQ ID NO: 38.

As used herein, a wild-type B. amyloliquefaciens “yitO” nucleic acid sequence encodes a wild-type B. amyloliquefaciens “YitO” polypeptide comprising SEQ ID NO: 36.

As used herein, a wild-type B. amyloliquefaciens “yitP” nucleic acid sequence encodes a wild-type B. amyloliquefaciens “YitP” polypeptide comprising SEQ ID NO: 34.

As used herein, a wild-type B. subtilis “sdpA” nucleic acid sequence (SEQ ID NO: 42) encodes a wild-type B. subtilis “SdpA” polypeptide comprising SEQ ID NO: 43.

As used herein, a wild-type B. subtilis “sdpB” nucleic acid sequence (SEQ ID NO: 44) encodes a wild-type B. subtilis “SdpB” polypeptide comprising SEQ ID NO: 45.

As used herein, a wild-type B. subtilis “sdpC” nucleic acid sequence (SEQ ID NO: 46) encodes a wild-type B. subtilis “SdpC” polypeptide comprising SEQ ID NO: 47.

As used herein, phrases such as “deficient in the production of a functional YitM protein”, “deficient in the production of a functional YitO protein”, “deficient in the production of a functional YitP protein”, and the like encompass a modified (mutant) Bacillus sp. cell that produces no detectable amount of the functional YitM protein, YitO protein and/or YitM protein (i.e., relative to the parental cell when grown/cultivated/fermented under identical conditions), or in the alternative, produces at least about 5% less to about 99% less of the one or more functional YitM, YitO and/or YitP proteins (i.e., relative to the parental cell when cultivated/fermented under identical conditions). For example, in certain embodiments, a modified Bacillus sp. cell of the disclosure comprises a genetic modification which deletes, disrupts, inactivates, etc. a regulatory region of the yitMOP operon, thereby rendering the modified cell deficient in the production of functional gene products (i.e., the YitM, YitO and/or YitP proteins). Thus, in certain embodiments, a promoter region sequence of the B. amyloliquefaciens yitMOP operon comprises a nucleotide sequence set forth in SEQ ID NO: 39, a promoter region sequence of the B. subtilis yitMOP operon comprises a nucleotide sequence set forth in SEQ ID NO: 40 and a promoter region sequence of the B. subtilis sdpABC operon comprises a nucleotide sequence set forth in SEQ ID NO: 41.

In certain other embodiments, a modified Bacillus sp. cell comprises a genetic modification which deletes, disrupts, inactivates or interferes with a regulatory region of the yitM gene (e.g., a promoter region sequence; SEQ ID NO: 39, SEQ ID NO: 40), thereby rendering the modified cell deficient in the production of functional YitM protein.

As used herein, a plasmid named “pUCyitMOP:spec” comprises a spectinomycin resistance (specR) marker gene flanked by loxP sites. More particularly, the plasmid pUCyitMOP:spec was constructed to replace the entire yitMOP operon in B. subtilis with a spectinomycin resistance (specR) marker.

As used herein, a “Topo-specR vector” comprises a spectinomycin adenyltransferase gene (specR) with its native promoter and terminator elements.

As used herein, a plasmid named “pUCsdp::tet” comprises upstream (5′) and downstream (3′) DNA sequences to replace the entire sdpABC operon in B. subtilis with a tetracycline (tetR) marker, flanked by loxP sites (herein, the “loxP-tetR-loxP cassette”), wherein removal of the tetracycline marker results in a deletion of the sdpABC operon.

As used herein, a “loxP-tetR-loxP cassette” comprises a loxP recombination sequence (Sauer, 1987, Sauer and Henderson 1988) that facilitates the loop out of the tetracycline resistance cassette later, after it has been used to select for chromosomal integration.

As used herein, a plasmid named “pUCLlacA-ganAB::tet” was used to delete ganAB genes of B. subtilis, which plasmid comprises homology regions upstream (5′) and downstream (3′) of ganAB and the loxP-tetR-loxP cassette.

As used herein, a “ME-3 protease” is a thermostable serine protease derived from Thermobifida cellulosilytica. For example, the T. cellulosilytica ME-3 protease and variants thereof are described in PCT Publication No. WO2018/118815, incorporated herein by referenced in its entirety.

As used herein, a B. subtilis strain named “ZM207” comprises a deleted alanine racemase gene (ΔalrA) and an introduced expression cassette encoding a ME-3 protease. More particularly, the introduced expression cassette comprises a promoter sequence (“P” positioned upstream (5′) and operably linked to an open reading frame (ORF) encoding the ME-3 protease which is upstream (5′) and operably linked to an ORF encoding the alanine racemase (alrA), wherein the cassette is integrated into the B. subtilis aprE locus (aprE::P-ME-3-alrA).

As used herein, a genetically modified B. subtilis strain named “ZM336” comprises an introduced ME-3 protease expression cassette and a deleted yitMOP operon (ΔyitMOP), wherein the deleted yitMOP operon was replaced with a spectinomycin resistance (specR) marker gene (i.e., ΔyitMOP). Thus, the ZM336 strain was constructed by introducing the deleted yitMOP operon (ΔyitMOP) into the parental ZM207 strain.

As used herein, a genetically modified B. subtilis strain named “ZM268” comprises a deletion of the yitMOP operon (ΔyitMOP) and a deletion of the sdpABC operon (ΔsdpABC). After deleting the yitMOP and sdpABC operons in an alanine racemase deleted strain (ΔalrA), an expression cassette encoding the ME-3 protease (aprE::P-ME-3-alrA) was introduced to obtain the ZM268 strain.

As used herein, a genetically modified B. subtilis strain named “ZM334” comprises a deletion of the sdpABC operon (ΔsdpABC) wherein the deleted sdpABC operon was replaced with a tetracycline resistance (tetR) marker gene (i.e., ΔsdpABC). Thus, the ZM334 strain was constructed by introducing the deleted sdpABC operon (ΔsdpABC) into the parental ZM207 strain.

As used herein, a genetically modified B. subtilis strain named “ZM434” comprises a deletion of the ΔyitM gene (ΔyitM), wherein the deleted yitM operon was replaced with an erythromycin resistance (ermC) marker gene (i.e., ΔyitM). Thus, the ZM434 strain was constructed by introducing the deleted yitM (ΔyitM) into the parental ZM207 strain.

As used herein, a “V42 protease” is a serine protease derived from Bacillus amyloliquefaciens subtilisin protease BPN′. For example, the V42 protease and variants thereof are described in PCT Publication No. WO2011/072099 (incorporated herein by referenced in its entirety).

As used herein, a parental B. subtilis strain named “CB24-12” comprises a deleted alanine racemase gene (ΔalrA) and an introduced expression cassette encoding the V42 protease. More particularly, the introduced expression cassette comprises a promoter sequence (“P4” positioned upstream (5′) and operably linked to an open reading frame (ORF) encoding the V42 protease which is upstream (5′) and operably linked to an ORF encoding the alanine racemase (alrA), wherein the cassette is integrated into the B. subtilis aprE locus (aprE::P4-V42-alrA).

As used herein, a genetically modified B. subtilis strain named “JL77” (i.e., derived from the CB24-12 parental strain), comprises the same expression cassette encoding the V42 protease, a deleted yitMOP operon (ΔyitMOP) and a deleted sdpABC operon (ΔsdpABC). For example, the yitMOP and sdpABC operons of the JL77 strain were deleted by homologous recombination with a cassette containing an antibiotic resistant marker. The yitMOP operon was replaced with a spectinomycin cassette flanked by loxP sequences. The spdABC operon was replaced by a tetracycline cassette flanked by loxP sequences. A transformation with a plasmid containing the cassette for expression of the Cre recombinase, the antibiotic resistant marker can be excised from the genome leaving one loxP sequence.

As used herein, the term “equivalent positions” mean the amino acid residue positions after alignment with a specified polypeptide sequence, or the nucleotide positions after alignment with a specified polynucleotide sequence.

The terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene, or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

As used herein, “disruption of a gene”, “gene disruption”, “inactivation of a gene” and “gene inactivation” are used interchangeably and refer broadly to any genetic modification that substantially prevents a host cell from producing a functional gene product (e.g., a YitM protein, a YitO protein, a YitP protein, etc.). Exemplary methods of gene disruptions include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and any combinations and variations thereof which disrupt/inactivate the target gene(s) and substantially reduce or prevent the production of the functional gene product (i.e., a protein).

As used herein, the combined term “expresses/produces”, as used in phrases such as “a modified cell expresses/produces an increased amount of a protein of interest relative to the parental cell”, the term “expresses/produces” is meant to include any steps involved in the expression and production of a protein of interest in a host cell of the disclosure.

As used herein, phrases such as “enhancing protein production” or “increasing protein production” refer to an increased amount of an endogenous protein or a heterologous protein produced by a host cell of the disclosure. In certain embodiments, the protein of interest is produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as protease activity, amylase activity, cellulase activity, hemicellulase activity and the like), or total extracellular protein produced as compared to the parental host cell.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in Bacillus. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence encoding a protein of the disclosure. Thus, in certain embodiments, a functional promoter sequence controls the expression of a gene encoding a protein disclosed herein. In other embodiments, a functional promoter sequence controls the expression of a heterologous gene (or endogenous gene) encoding a protein of interest in a Bacillus sp. cell.

As defined herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As defined herein, the term “introducing”, as used in phrases such as “introducing into a bacterial cell” or “introducing into a Bacillus sp. cell” at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like (e.g., see Ferrari et al., 1989).

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). For example, in certain embodiments of the disclosure, a parental Bacillus sp. cell is modified (e.g., transformed) by introducing into the parental cell a polynucleotide construct comprising a promoter operably linked to a nucleic acid sequence encoding a protein of interest, thereby resulting in a modified Bacillus sp. (daughter) host cell derived from the parental cell.

As used herein, “transformation” refers to introducing an exogenous DNA into a host cell so that the DNA is maintained as a chromosomal integrant or a self-replicating extra-chromosomal vector. As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, conjugation, and transduction (See e.g., Ferrari et al., 1989).

As used herein “an incoming sequence” refers to a DNA sequence that is introduced into the Bacillus chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the Bacillus chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the Bacillus chromosome a DNA construct is integrated and directs what part of the Bacillus chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the phrases “selectable marker gene” or “selectable marker encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient. Examples of such selectable markers include, but are not limited to, antimicrobials. Thus, a selectable marker refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., amp^{R}, phleo^{R}, spec^{R}, kan^{R}, ery^{R}, tet^{R}, cmp^{R} and neo^{R} (see e.g., Guerot-Fleury, 1995; Palmeros et al., 2000; and Trieu-Cuot et al., 1983). In some embodiments, the present invention provides a chloramphenicol resistance gene (e.g., the gene present on pC194, as well as the resistance gene present in the Bacillus sp. genome). This resistance gene is particularly useful in the present invention, as well as in embodiments involving chromosomal amplification of chromosomally integrated cassettes and integrative plasmids (see e.g., Albertini and Galizzi, 1985; Stahl and Ferrari, 1984). Other markers useful in accordance with the invention include, but are not limited to auxotrophic markers, such as serine, lysine, tryptophan; and detection markers, such as β-galactosidase or fluorescent proteins.

As defined herein, a host cell “genome”, a bacterial cell “genome”, or a Bacillus sp. “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the term “expression cassette” refers to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). In some embodiments the targeting vectors include elements to increase homologous recombination with the chromosome including but not limited to RNA-guided endonucleases, DNA-guided endonucleases, and recombinases. The ends can be closed such that the targeting vector forms a closed circle, such as, for example, insertion into a vector.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

As used herein, the term “protein of interest” (abbreviated “POI”) refers to a protein of interest that is desired to be expressed in a Bacillus sp. host cell. Thus, as used herein, a POI may be an enzyme, a substrate-binding protein, a surface-active protein, a structural protein, a receptor protein, and the like. In certain embodiments, a modified cell of the disclosure produces an increased amount of a heterologous POI or an increased amount of an endogenous POI, relative to the parental cell. In particular embodiments, an increased amount of a POI produced by a modified cell of the disclosure is at least a 0.5% increase, at least a 1.0% increase, at least a 5.0% increase, or a greater than 5.0% increase, relative to the parental cell.

Similarly, as defined herein, a “gene of interest” (abbreviated “GOP”) refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous” nucleic acid construct or a “heterologous” nucleic acid sequence has a portion of the sequence which is not native to the cell in which it is expressed.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to the another specified material or composition.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wis., USA 53711) (Needleman and Wunsch, (1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, “orthologue” and “orthologous genes” refer to genes in different species that have evolved from a common ancestral gene (i.e., a homologous gene) by speciation. Typically, orthologues retain the same function during the course of evolution. Identification of orthologues finds use in the reliable prediction of gene function in newly sequenced genomes.

As used herein, “paralog” and “paralogous genes” refer to genes that are related by duplication within a genome. While orthologues retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, “homology” refers to sequence similarity or identity, with identity being preferred. This homology is determined using standard techniques known in the art (see e.g., Smith and Waterman, 1981; Needleman and Wunsch, 1970; Pearson and Lipman, 1988; programs such as GAP, BESTFIT, FASTA, and TFASTA in the Wisconsin Genetics Software Package (Genetics Computer Group, Madison, Wis.) and Devereux et. al., 1984).

As used herein, the term “hybridization” refers to the process by which a strand of nucleic acid joins with a complementary strand through base pairing, as known in the art. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tₘ) of the nucleic acid binding complex or probe. For example, “maximum stringency” typically occurs at about Tₘ ⁻5° C. (5° below the Tₘ of the probe); “high stringency” at about 5-10° C. below the Tₘ; “intermediate stringency” at about 10-20° C. below the Tₘ of the probe; and “low stringency” at about 20-25° C. below the Tₘ.

Functionally, maximum stringency conditions may be used to identify sequences having strict identity or near-strict identity with the hybridization probe; while an intermediate or low stringency hybridization can be used to identify or detect polynucleotide sequence homologs. Moderate and high stringency hybridization conditions are well known in the art. An example of high stringency conditions includes hybridization at about 42° C. in 50% formamide, 5×SSC, 5×Denhardt's solution, 0.5% SDS and 100 pg/ml denatured carrier DNA, followed by washing two times in 2×SSC and 0.5% SDS at room temperature (RT) and two additional times in 0.1×SSC and 0.5% SDS at 42° C. An example of moderate stringent conditions including overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denatured sheared salmon sperm DNA, followed by washing the filters in 1×SSC at about 37-50° C. Those of skill in the art know how to adjust the temperature, ionic strength, etc. as necessary to accommodate factors such as probe length and the like.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. “Recombination”, “recombining” or generating a “recombined” nucleic acid is the assembly of two or more nucleic acid fragments wherein the assembly gives rise to a chimeric gene.

As used herein, a “flanking sequence” refers to any sequence that is either upstream (5′) or downstream (3′) of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked. In some embodiments, the homology boxes are directly flanking each other and lacking an intervene sequence (e.g. for genes D-E-F the construct D-F) such that if the construct recombines within the genome gene E will be removed from the genome.

### II. CANNIBALISM CELL STRESS RESPONSE

In their natural environment, micro-organisms must constantly compete for nutrients. In order to defend their habitat from invading species, many bacteria produce and secrete antimicrobial peptides (AMPs) that interfere with the integrity and/or biosynthesis of the cell envelope (e.g., AMP action leads to an arrest in cell growth and often to cell lysis (Silver, 2003; Silver, 2006)). Thus, to defend against such antimicrobial peptide (AMP) attacks, many bacteria induce a complex cell envelope stress response. For example, when faced with carbon source limitations, B. subtilis initiates a survival strategy called sporulation, leading to the formation of highly resistant endospores that allow B. subtilis to survive long periods of starvation. In order to avoid commitment to this energy-demanding and irreversible process, B. subtilis employs another strategy called “cannibalism” to delay sporulation for as long as possible (Gonzalez-Pastor et al., 2003). Cannibalism in B. subtilis generally involves the production and secretion of two known cannibalism toxins (AMPs), the sporulation delaying protein (SDP) and the sporulation killing factor (SKF), which cannibalism toxin proteins are able to lyse sensitive siblings. Thus, the lysed sibling cells are thought to then provide nutrients for the cannibals to slow down, or even prevent them from entering sporulation.

For example, as generally described by Gonzalez-Pastor et al. (2003), the B. subtilis skf operon comprises eight (8) genes (i.e., skfABCDEFGH), wherein the skfA gene encodes the AMP toxin SKF; and the B. subtilis sdp operon comprises three (3) genes (i.e., sdpABC), wherein sdpC gene encodes the AMP toxin SPD. A more recent study of the spdABC operon by Perez Morales et al. (2013) suggests that the encoded SdpA and SdpB proteins are essential for the post-translational processing of the immature SpdC protein into the mature and active SDP toxin protein, which is secreted extracellularly. Wang et al. (2014) studied the effects of single and combined deletions of certain cell lysis genes in B. subtilis (i.e., skfA, sdpC, xpf or lytC), wherein the highest numbers of intact cells were detected in cultures of the quadruple-mutant (ΔlytCΔsdpCΔxpfΔskfA). As concluded by Wang et al. (2014), the production of intracellular and secreted recombinant proteins (nattokinase and β-galactosidase, respectively) was significantly elevated in cultures of the quadruple mutant LM2531 (ΔlytCΔsdpCΔxpfΔskfA) by the deletion of lytC which encodes a major PG hydrolase, prophage-encoded xpf and cannibalism factors skfA and sdpC.

As described herein and presented below in the Examples section, Applicant has identified a novel cannibalism operon in B. subtilis named yitMOP, which operon encodes a YitM protein (SEQ ID NO: 2), a YitO protein (SEQ ID NO: 4) and a YitP protein (SEQ ID NO: 6). More specifically, Applicant identified the novel yitMOP operon as a highly expressed transcript in a transcriptome analysis of a B. subtilis strain expressing FNA protease.

Thus, as further described in the Example 3 below, Applicant constructed a parental B. subtilis cell (strain) named ZM207, comprising an introduced protease (ME-3) expression cassette, and modified B. subtilis cells (strains) thereof named ZM334, ZM336, and ZM434; wherein the ZM334 cell comprises the same protease (ME-3) expression cassette and a deleted sdpABC operon (ΔsdpABC), the ZM336 cell comprises the ME-3 protease expression cassette and a deleted yitMOP operon (ΔyitMOP), and the ZM434 cell comprises the ME-3 protease expression cassette and a deletion of the yitM gene (ΔyitM). The ZM268 cell comprises the ME-3 protease expression cassette, a deleted yitMOP operon (ΔyitMOP) and a deleted sdpABC operon (ΔsdpABC), and was constructed by introducing the protease (ME-3) expression cassette into a host with a deletion of yitMOP operon (ΔyitMOP), a deleted sdpABC operon (ΔsdpABC), and a deletion of the alanine racemase gene (ΔalrA).

As presented in Example 3, the parental (ZM207; control) and modified B. subtilis cells (ZM334, ZM336, ZM268 and ZM434) where fermented in shake flasks under identical conditions in Grant's II medium at 42° C. for forty (40) hours. Subsequently, fermentation supernatants were taken and the ME-3 protease activity (TABLE 15) was measured. For example, as shown in TABLE 15, both the ZM334 (ΔsdpABC) strain and the ZM336 (ΔyitMOP) strain produced an increased amount of the ME-3 protease relative to the ZM207 (control) strain. Similarly, the ZM268 strain (TABLE 15), comprising a combined deletion of the yitMOP and sdpABC operons (i.e., ΔyitMOP+ΔsdpABC) produces an increased amount of the protease relative to the ZM207 parental strain, the ZM334 strain (ΔsdpABC) and the ZM336 strain (ΔyitMOP). In addition, it was surprisingly observed (TABLE 15) that disruption or deletion of the yitM ORF alone was sufficient to elicit a beneficial phenotype of increased protein production, as the ZM434 (ΔyitM) strain produced slightly more ME-3 protease than the ZM336 (ΔyitMOP) strain.

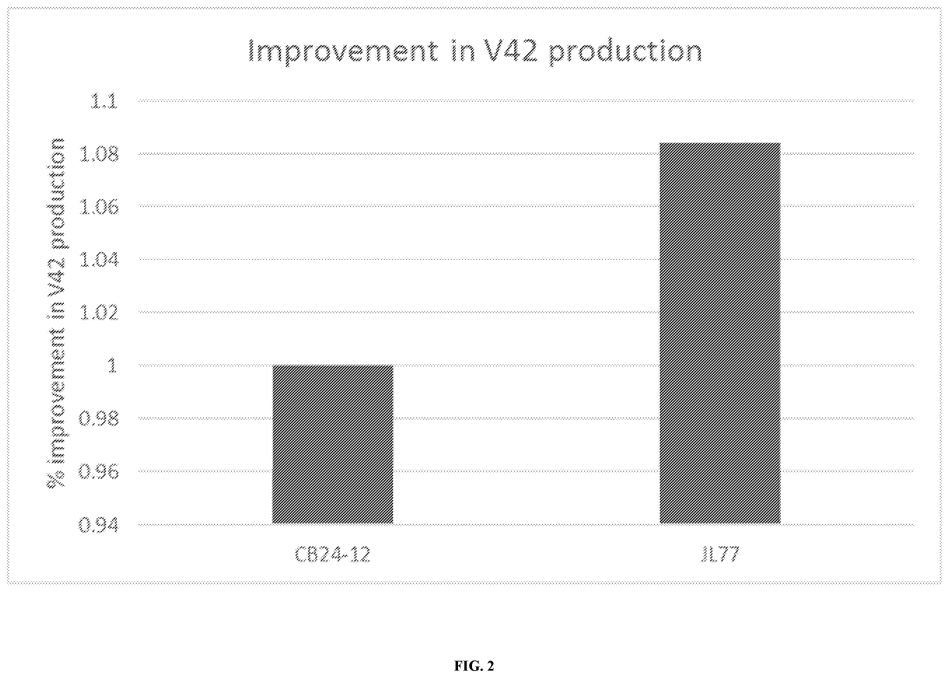

Likewise, as presented and described below in Example 4, Applicant further constructed a parental B. subtilis cell (strain) named CB24-12, comprising an introduced protease (V42) expression cassette, and a modified B. subtilis daughter cell named JL77, comprising the same V42 protease expression cassette and a combined deletion of the yitMOP operon and the sdpABC operon (ΔyitMOP+ΔsdpABC). As described in Example 4, the B. subtilis CB24-12 and JL77 strains were evaluated for V42 protease production under large scale fermentation conditions. For example, as presented in FIG. 2, the CB24-12 and L77 strains were tested in fermentors under identical standard conditions, wherein each strain was run in duplicate, and the average improvement in V42 protease production at the end of fermentation is shown. Thus, as shown in FIG. 2, the average increase in V42 protease production at the end of fermentation for the JL77 strain was approximately 10% increased relative to the amount of V42 protease produced by the CB24-12 strain at the end of fermentation.

These findings demonstrate that genetic modifications of the yitMOP operon in Bacillus sp. cells result in an enhanced protein productivity phenotype relative to parental Bacillus sp. cells (i.e., comprising a native yitMOP operon). In addition, these findings demonstrate that the combined genetic modification of the yitMOP operon and the sdpABC operon in Bacillus sp. cells further enhances the observed protein productivity phenotype relative to modified Bacillus sp. cells comprising a genetic modification of the yitMOP operon alone (see, Example 3, TABLE 15 and Example 4, FIG. 2).

Likewise, it was surprisingly observed herein that Bacillus sp. cells comprising a genetic modification of the yitM gene alone demonstrated an enhanced protein productivity phenotype relative to the Bacillus sp. cells comprising the combined genetic modifications of the yitMOP and sdpABC operons (ΔyitMOP+ΔsdpABC; Example 3, TABLE 15).

### III. MOLECULAR BIOLOGY

As set forth above, certain embodiments of the disclosure are related to modified Bacillus sp. cells derived from parental Bacillus sp. cells comprising a native yitMOP operon. In particular embodiments, a modified Bacillus sp. cell comprises a modified yitMOP operon. In certain other embodiments, a modified Bacillus sp. cell comprises a disrupted or deleted yitM gene. In certain other embodiments, a modified Bacillus sp. cell of the disclosure further comprises a deleted spdABC operon. Certain embodiments are related to compositions and methods for genetically modifying a parental Bacillus sp. cell to generate modified B. licheniformis (daughter) cell therefrom.

Thus, certain embodiments of the disclosure are related to methods for genetically modifying Bacillus sp. cells, including, but not limited to, (a) the introduction, substitution, or removal of one or more nucleotides in a gene (or an ORF thereof), or the introduction, substitution, or removal of one or more nucleotides in a regulatory element required for the transcription or translation of the gene (or ORF thereof), (b) a gene disruption, (c) a gene conversion, (d) a gene deletion, (e) a gene down-regulation, (f) site specific mutagenesis and/or (g) random mutagenesis. For example, as used herein a genetic modification includes, but is not limited to, a modification of one or more genes from of a Bacillus sp. cell (e.g., one or more genes of the yitMOP operon and/or the spdABC operon).

Thus, in certain embodiments, a modified Bacillus cell of the disclosure is constructed by reducing or eliminating the expression of a gene, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified Bacillus sp. cell is constructed by gene deletion to eliminate or reduce the expression of at least one of the aforementioned genes of the disclosure. Gene deletion techniques enable the partial or complete removal of the gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a Bacillus cell, for example, on a temperature-sensitive plasmid, such as pE194, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers (see, e.g., Perego, 1993). Thus, a person of skill in the art (e.g., by reference to the yitM, yitO and yitP (nucleic acid) sequences and the encoded YitM, YitO and YitP protein sequences thereof), may readily identify nucleotide regions in the gene's coding sequence and/or the gene's non-coding sequence suitable for complete or partial deletion.

In other embodiments, a modified Bacillus cell of the disclosure is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art (e.g., see, Botstein and Shortie, 1985; Lo et al., 1985; Higuchi et al., 1988; Shimada, 1996; Ho et al., 1989; Horton et al., 1989 and Sarkar and Sommer, 1990). Thus, in certain embodiments, a gene of the disclosure is inactivated by complete or partial deletion.

In another embodiment, a modified Bacillus cell is constructed by the process of gene conversion (e.g., see Iglesias and Trautner, 1983). For example, in the gene conversion method, a nucleic acid sequence corresponding to the gene(s) is mutagenized in vitro to produce a defective nucleic acid sequence, which is then transformed into the parental Bacillus cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene (Perego, 1993). Alternatively, the defective nucleic acid sequence may contain an insertion, substitution, or deletion of one or more nucleotides of the gene, as described below.

In other embodiments, a modified Bacillus cell is constructed by established anti-sense techniques using a nucleotide sequence complementary to the nucleic acid sequence of the gene (Parish and Stoker, 1997). More specifically, expression of the gene by a Bacillus cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a modified Bacillus cell is produced/constructed via CRISPR-Cas9 editing. For example, a gene encoding the YitM protein can be disrupted (or deleted or down-regulated) by means of nucleic acid guided endonucleases, that find their target DNA by binding either a guide RNA (e.g., Cas9) and Cpfl or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the Bacillus cell and a terminator active in Bacillus cell, thereby creating a Bacillus Cas9 expression cassette. Likewise, one or more target sites unique to the gene of interest are readily identified by a person skilled in the art. For example, to build a DNA construct encoding a gRNA-directed to a target site within the gene of interest using Streptococcus pyogenes Cas9, the variable targeting domain (VT) will comprise nucleotides of the target site which are 5′ of the (PAM) proto-spacer adjacent motif (NGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Bacillus expression cassette for the gRNA is created by operably linking the DNA encoding the gRNA to a promoter active in Bacillus cells and a terminator active in Bacillus cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500-bp 5′ of targeted gene can be fused to about 500-bp 3′ of the targeted gene to generate an editing template, which template is used by the Bacillus host's machinery to repair the DNA break generated by the RGEN.

The Cas9 expression cassette, the gRNA expression cassette and the editing template can be co-delivered to the cells using many different methods. The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies.

In yet other embodiments, a modified Bacillus cell is constructed by random or specific mutagenesis using methods well known in the art, including, but not limited to, chemical mutagenesis (see, e.g., Hopwood, 1970) and transposition (see, e.g., Youngman et al., 1983). Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

International PCT Publication No. WO2003/083125 discloses methods for modifying Bacillus cells, such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., E. coli and Bacillus sp.) (e.g., Ferrari et al., 1989; Saunders et al., 1984; Hoch et al., 1967; Mann et al., 1986; Holubova, 1985; Chang et al., 1979; Vorobjeva et al., 1980; Smith et al., 1986; Fisher et al., 1981 and McDonald, 1984). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art (e.g., Stahl et al., 1984; Palmeros et al., 2000). In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Bacillus cells are generally known on one of skill in the art. Promoter sequences of the disclosure are generally chosen so that they are functional in the Bacillus cells. Certain exemplary Bacillus promoter sequences include, but are not limited to, the B. subtilis alkaline protease (aprE) promoter, the α-amylase promoter of B. subtilis, the α-amylase promoter of B. amyloliquefaciens, the neutral protease (nprE) promoter from B. subtilis, a mutant aprE promoter (e.g., PCT Publication No. WO2001/51643) or any other promoter from B licheniformis or other related Bacilli. Methods for screening and creating promoter libraries with a range of activities (promoter strength) in Bacillus cells is describe in PCT Publication No. WO2003/089604.

### IV. CULTURING MODIFIED CELLS FOR PRODUCTION OF A PROTEIN OF INTEREST

As generally described above, certain embodiments are related to compositions and methods for constructing and obtaining Bacillus cells having increased protein production phenotypes. Thus, certain embodiments are related to methods of producing proteins of interest in Bacillus cells by fermenting the cells in a suitable medium. Fermentation methods well known in the art can be applied to ferment the parental and modified (daughter) Bacillus cells of the disclosure.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO2. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Bacillus cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO2. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Bacillus cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

### V. PROTEINS OF INTEREST

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

For example, in certain embodiments, a modified Bacillus cell of the disclosure produces at least about 0.1% more, at least about 0.5% more, at least about 1% more, at least about 5% more, at least about 6% more, at least about 7% more, at least about 8% more, at least about 9% more, or at least about 10% or more of a POI, relative to its unmodified (parental) cell.

In certain embodiments, a modified Bacillus cell of the disclosure exhibits an increased specific productivity (Qp) of a POI relative the (unmodified) parental cell. For example, the detection of specific productivity (Qp) is a suitable method for evaluating protein production. The specific productivity (Qp) can be determined using the following equation:

“Qp=gP/gDCW·hr”

wherein, “gP” is grams of protein produced in the tank; “gDCW” is grams of dry cell weight (DCW) in the tank and “hr” is fermentation time in hours from the time of inoculation, which includes the time of production as well as growth time.

Thus, in certain other embodiments, a modified Bacillus cell of the disclosure comprises a specific productivity (Qp) increase of at least about 0.1%, at least about 1%, at least about 5%, at least about 6%, at least about 7%, at least about 8%, at least about 9%, or at least about 10% or more, relative to the unmodified (parental) cell.

In certain embodiments, a POI or a variant POI thereof is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5 or EC 6.

In certain other embodiments, a modified Bacillus cell of the disclosure comprises an expression construct encoding an amylase. A wide variety of amylase enzymes and variants thereof are known to one skilled in the art. For example, International PCT Publication NO. WO2006/037484 and WO 2006/037483 describe variant α-amylases having improved solvent stability, PCT Publication No. WO1994/18314 discloses oxidatively stable α-amylase variants, PCT Publication No. WO1999/19467, WO2000/29560 and WO2000/60059 disclose Termamyl-like α-amylase variants, PCT Publication No. WO2008/112459 discloses α-amylase variants derived from Bacillus sp. number 707, PCT Publication No. WO1999/43794 discloses maltogenic α-amylase variants, PCT Publication No. WO1990/11352 discloses hyper-thermostable α-amylase variants, PCT Publication No. WO2006/089107 discloses α-amylase variants having granular starch hydrolyzing activity, and the like.

There are various assays known to those of ordinary skill in the art for detecting and measuring activity of intracellularly and extracellularly expressed proteins.

PCT Publication No. WO2014/164777 discloses Ceralpha α-amylase activity assays useful for detecting amylase activities described herein.

### V. EXEMPLARY EMBODIMENTS

Non-limiting embodiments of the disclosure include, but are not limited to:

1. A method for producing an increased amount of an endogenous protein of interest (POI) in a modified Bacillus sp. cell comprising (a) obtaining a parental Bacillus sp. cell producing an endogenous POI, (b) genetically modifying the parental Bacillus sp. cell by deleting or disrupting the yitMOP operon, and (c) fermenting the modified cell under suitable conditions for the production of the POI, wherein the modified cell produces an increased amount of the endogenous POI relative to the parental cell when fermented under the same conditions.

2. A method for producing an increased amount of an endogenous protein of interest (POI) in a modified Bacillus sp. cell comprising (a) obtaining a parental Bacillus sp. cell producing an endogenous POI, (b) genetically modifying the parental Bacillus sp. by deleting or disrupting the yitMOP operon and the sdpABC operon, and (c) fermenting the modified cell under suitable conditions for the production of the POI, wherein the modified cell produces an increased amount of the endogenous POI relative to the parental cell when fermented under the same conditions.

3. A method for producing an increased amount of an endogenous protein of interest (POI) in a modified Bacillus sp. cell comprising (a) obtaining a parental Bacillus sp. cell producing an endogenous POI, (b) genetically modifying the parental Bacillus sp. cell by deleting or disrupting a regulatory region of the yitMOP operon, thereby rendering the modified cell deficient in the production of functional YitM, YitO and/or YitP proteins, and (c) fermenting the modified cell under suitable conditions for the production of the POI, wherein the modified cell produces an increased amount of the endogenous POI relative to the parental cell when fermented under the same conditions.

4. A method for producing an increased amount of an endogenous protein of interest (POI) in a modified Bacillus sp. cell comprising (a) obtaining a parental Bacillus sp. cell producing an endogenous POI, (b) genetically modifying the parental cell by disrupting or deleting the yitM gene, and (c) fermenting the modified cell under suitable conditions for the production of the POI, wherein the modified cell produces an increased amount of the endogenous POI relative to the parental cell when fermented under the same conditions.

5. A method for producing an increased amount of an endogenous protein of interest (POI) in a modified Bacillus sp. cell comprising (a) obtaining a parental Bacillus sp. cell producing an endogenous POI, (b) genetically modifying the parental Bacillus sp. cell by deleting or disrupting a regulatory region of the yitM gene, thereby rendering the modified cell deficient in the production of functional YitM protein, and (c) fermenting the modified cell under suitable conditions for the production of the POI, wherein the modified cell produces an increased amount of the endogenous POI relative to the parental cell when fermented under the same conditions.

6. The method of any one of embodiments 1-5, wherein the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus and B. thuringiensis.

7. The method of embodiment 6, wherein the Bacillus sp. cell is B. subtilis.

8. The method of embodiment 6, wherein the Bacillus sp. cell is B. amyloliquefaciens.

9. The method of any one of embodiments 1-5, wherein the POI is an enzyme.

10. The method of embodiment 7, wherein the enzyme is a protease or an amylase.

11. The method of any one of embodiments 1-3, wherein the yitMOP operon encodes a YitM polypeptide comprising at least 60% to about 99% sequence identity to SEQ ID NO: 2 or SEQ ID NO: 38, a YitO polypeptide comprising at least 60% to about 99% sequence identity to SEQ ID NO: 4 or SEQ ID NO: 36, and YitP polypeptide comprising at least 60% to about 99% sequence identity to SEQ ID NO: 6 or SEQ ID NO: 34.

12. The method of embodiment 4 or embodiment 5, wherein the yitM gene comprises at least 60% to about 99% sequence identity to the yitM gene of SEQ ID NO: 1 or SEQ ID NO: 37.

13. The method of embodiment 4 or embodiment 5, wherein the yitM gene encodes a YitM polypeptide comprising at least 60% to about 99% sequence identity to the YitM polypeptide of SEQ ID NO: 2 or SEQ ID NO: 38.

14. The method of any one of embodiments 3-5, wherein the modified cell further comprises a deletion or disruption of the sdpABC operon.

15. A method for producing an increased amount of a heterologous protein of interest (POI) in a modified Bacillus sp. cell comprising (a) constructing a parental Bacillus sp. cell comprising an introduced expression cassette encoding a heterologous POI, (b) genetically modifying the parental cell by deleting or disrupting the yitMOP operon, and (c) fermenting the modified cell under suitable conditions for the production of the POI, wherein the modified cell produces an increased amount of the heterologous POI relative to the parental cell when fermented under the same conditions.

16. A method for producing an increased amount of a heterologous protein of interest (POI) in a modified Bacillus sp. cell comprising (a) constructing a parental Bacillus sp. cell comprising an introduced expression cassette encoding a heterologous POI, (b) genetically modifying the parental Bacillus sp. by deleting or disrupting the yitMOP operon and the sdpABC operon, and (c) fermenting the modified cell under suitable conditions for the production of the POI, wherein the modified cell produces an increased amount of the heterologous POI relative to the parental cell when fermented under the same conditions.

17. A method for producing an increased amount of a heterologous protein of interest (POI) in a modified Bacillus sp. cell comprising (a) constructing a parental Bacillus sp. cell comprising an introduced expression cassette encoding a heterologous POI, (b) genetically modifying the parental Bacillus sp. cell by deleting or disrupting a regulatory region of the yitMOP operon, thereby rendering the modified cell deficient in the production of functional YitM, YitO and/or YitP proteins, and (c) fermenting the modified cell under suitable conditions for the production of the POI, wherein the modified cell produces an increased amount of the heterologous POI relative to the parental cell when fermented under the same conditions.

18. A method for producing an increased amount of a heterologous protein of interest (POI) in a modified Bacillus sp. cell comprising (a) constructing a parental Bacillus sp. cell comprising an introduced expression cassette encoding a heterologous POI, (b) genetically modifying the parental cell by disrupting or deleting the yitM gene, and (c) fermenting the modified cell under suitable conditions for the production of the POI, wherein the modified cell produces an increased amount of the heterologous POI relative to the parental cell when fermented under the same conditions.

19. A method for producing an increased amount of a heterologous protein of interest (POI) in a modified Bacillus sp. cell comprising (a) constructing a parental Bacillus sp. cell comprising an introduced expression cassette encoding a heterologous POI, (b) genetically modifying the parental Bacillus sp. cell by deleting or disrupting a regulatory region of the yitM gene, thereby rendering the modified cell deficient in the production of functional YitM protein, and (c) fermenting the modified cell under suitable conditions for the production of the POI, wherein the modified cell produces an increased amount of the heterologous POI relative to the parental cell when fermented under the same conditions.

20. The method of any one of embodiments 15-19, wherein steps (a) and (b) are performed simultaneously.

21. The method of any one of embodiments 15-19, wherein the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus and B. thuringiensis.

22. The method of embodiment 21, wherein the Bacillus sp. cell is B. subtilis.

23. The method of embodiment 21, wherein the Bacillus sp. cell is B. amyloliquefaciens.

24. The method of any one of embodiments 15-19, wherein the heterologous POI is an enzyme.

25. The method of embodiment 24, wherein the enzyme is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.

26. The method of any one of embodiments 15-17, wherein the yitMOP operon encodes a YitM polypeptide comprising at least 80% sequence identity to SEQ ID NO: 2 or SEQ ID NO: 38, a YitO polypeptide comprising at least 80% sequence identity to SEQ ID NO: 4 or SEQ ID NO: 36, and YitP polypeptide comprising at least 80% sequence identity to SEQ ID NO: 6 or SEQ ID NO: 34.

27. The method of embodiment 18 or embodiment 19, wherein the yitM gene comprises at least 60% to about 99% sequence identity to the yitM gene of SEQ ID NO: 1 or SEQ ID NO: 37.

28. The method of embodiment 18 or embodiment 19, wherein the yitM gene encodes a YitM polypeptide comprising at least 60% to about 99% sequence identity to the YitM polypeptide of SEQ ID NO: 2 or SEQ ID NO: 38.

29. The method of any one of embodiments 17-19, wherein the modified cell further comprises a deletion or disruption of the sdpABC operon.

30. A genetically modified Bacillus sp. cell produced according to the methods of any one of embodiments 1-5, or a genetically modified Bacillus sp. cell produced according to the methods of any one of embodiments 15-19.

31. A genetically modified Bacillus sp. cell derived from a parental Bacillus sp. cell producing an endogenous protein of interest (POI), wherein the modified cell comprises a deleted or disrupted yitMOP operon, wherein the modified cell produces an increased amount of the endogenous POI relative to the parental cell when fermented under the same conditions for the production of the POI.

32. A genetically modified Bacillus sp. cell derived from a parental Bacillus sp. cell producing an endogenous protein of interest (POI), wherein the modified cell comprises a deleted or disrupted yitMOP operon and a deleted or disrupted sdpABC operon, wherein the modified cell produces an increased amount of the endogenous POI relative to the parental cell when fermented under the same conditions for the production of the POI.

33. A genetically modified Bacillus sp. cell derived from a parental Bacillus sp. cell producing an endogenous protein of interest (POI), wherein the modified cell comprises a deleted or disrupted regulatory region of the yitMOP operon rendering the modified cell deficient in the production of functional YitM, YitO and/or YitP proteins, wherein the modified cell produces an increased amount of the endogenous POI relative to the parental cell when fermented under the same conditions for the production of the POI.

34. A genetically modified Bacillus sp. cell derived from a parental Bacillus sp. cell producing an endogenous protein of interest (POI), wherein the modified cell comprises a deleted or disrupted yitM gene, wherein the modified cell produces an increased amount of the endogenous POI relative to the parental cell when fermented under the same conditions.

35. A genetically modified Bacillus sp. cell derived from a parental Bacillus sp. cell producing an endogenous protein of interest (POI), wherein the modified cell comprises a deleted or disrupted regulatory region of the yitM gene rendering the modified cell deficient in the production of functional YitM protein, wherein the modified cell produces an increased amount of the endogenous POI relative to the parental cell when fermented under the same conditions.

36. The modified cell of any one of embodiments 31-35, wherein the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus and B. thuringiensis.

37. The modified cell of embodiment 36, wherein the Bacillus sp. cell is B. subtilis.

38. The modified cell embodiments 36, wherein the Bacillus sp. cell is B. amyloliquefaciens.

39. The modified cell of any one of embodiments 29-33, wherein the POI is an enzyme.

40. The modified cell of embodiment 39, wherein the enzyme is a protease or an amylase.

41. The modified cell of any one of embodiments 31-33, wherein the yitMOP operon encodes a YitM polypeptide comprising at least 60% to about 99% sequence identity to SEQ ID NO: 2 or SEQ ID NO: 38, a YitO polypeptide comprising at least 60% to about 99% sequence identity to SEQ ID NO: 4 or SEQ ID NO: 36, and YitP polypeptide comprising at least 60% to about 99% sequence identity to SEQ ID NO: 6 or SEQ ID NO: 34.

42. The modified cell of embodiment 34 or embodiment 35, wherein the yitM gene comprises at least 60% to about 99% sequence identity to the yitM gene of SEQ ID NO: 1 or SEQ ID NO: 37.

43. The modified cell of embodiment 34 or embodiment 35, wherein the yitM gene encodes a YitM polypeptide comprising at least 60% to about 99% sequence identity to the YitM polypeptide of SEQ ID NO: 2 or SEQ ID NO: 38.

44. The modified cell of any one of embodiments 33-35, wherein the modified cell further comprises a deletion or disruption of the sdpABC operon.

45. A genetically modified Bacillus sp. cell derived from a parental Bacillus sp. cell comprising an introduced expression cassette encoding a heterologous protein of interest (POI), wherein the modified cell comprises a deleted or disrupted yitMOP operon, wherein the modified cell produces an increased amount of the heterologous POI relative to the parental cell when fermented under the same conditions for the production of the POI.

46. A genetically modified Bacillus sp. cell derived from a parental Bacillus sp. cell comprising an introduced expression cassette encoding a heterologous protein of interest (POI), wherein the modified cell comprises a deleted or disrupted yitMOP operon and a deleted or disrupted sdpABC operon, wherein the modified cell produces an increased amount of the endogenous POI relative to the parental cell when fermented under the same conditions for the production of the POI.

47. A genetically modified Bacillus sp. cell derived from a parental Bacillus sp. cell comprising an introduced expression cassette encoding a heterologous protein of interest (POI), wherein the modified cell comprises a deleted or disrupted regulatory region of the yitMOP operon rendering the modified cell deficient in the production of functional YitM, YitO and/or YitP proteins, wherein the modified cell produces an increased amount of the endogenous POI relative to the parental cell when fermented under the same conditions for the production of the POI.

48. A genetically modified Bacillus sp. cell derived from a parental Bacillus sp. cell comprising an introduced expression cassette encoding a heterologous protein of interest (POI), wherein the modified cell comprises a deleted or disrupted yitM gene, wherein the modified cell produces an increased amount of the endogenous POI relative to the parental cell when fermented under the same conditions.

49. A genetically modified Bacillus sp. cell derived from a parental Bacillus sp. cell comprising an introduced expression cassette encoding a heterologous protein of interest (POI), wherein the modified cell comprises a deleted or disrupted regulatory region of the yitM gene rendering the modified cell deficient in the production of functional YitM protein, wherein the modified cell produces an increased amount of the endogenous POI relative to the parental cell when fermented under the same conditions.

50. The modified cell of any one of embodiments 45-49, wherein the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus and B. thuringiensis.

51. The modified cell of embodiment 50, wherein the Bacillus sp. cell is B. subtilis.

52. The modified cell embodiment 50, wherein the Bacillus sp. cell is B. amyloliquefaciens.

53. The modified cell of any one of embodiments 45-49, wherein the POI is an enzyme.

54. The modified cell of embodiment 53, wherein the wherein the enzyme is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.

55. The modified cell of any one of embodiments 45-47, wherein the yitMOP operon encodes a YitM polypeptide comprising at least 60% to about 99% sequence identity to SEQ ID NO: 2 or SEQ ID NO: 38, a YitO polypeptide comprising at least 60% to about 99% % sequence identity to SEQ ID NO: 4 or SEQ ID NO: 36, and YitP polypeptide comprising at least 60% to about 99% sequence identity to SEQ ID NO: 6 or SEQ ID NO: 34.

56. The modified cell of embodiment 48 or embodiment 49, wherein the yitM gene comprises at least 80% sequence identity to the yitM gene of SEQ ID NO: 1 or SEQ ID NO: 37.

57. The modified cell of embodiment 48 or embodiment 49, wherein the yitM gene encodes a YitM polypeptide comprising at least 80% sequence identity to the YitM polypeptide of SEQ ID NO: 2 or SEQ ID NO: 38.

58. The modified cell of any one of embodiments 47-49, wherein the modified cell further comprises a deletion or disruption of the sdpABC operon.

59. The method of any one of embodiments 3, 5, 17 or 19, wherein the deleted or disrupted regulatory region is a promoter sequence.

60. The method of embodiment 59, wherein the promoter sequence comprises at least 60% to about 99% sequence identity to a nucleotide sequence selected from SEQ ID NO: 39, SEQ ID NO: 39 and SEQ ID NO: 40.

61. The modified cell of any one of embodiments 33, 35, 47 or 49, wherein the deleted or disrupted regulatory region is a promoter sequence.

62. The modified cell of embodiment 61, wherein the promoter sequence comprises at least 60% to about 99% sequence identity to a nucleotide sequence selected from SEQ ID NO: 39, SEQ ID NO: 39 and SEQ ID NO: 40.

### EXAMPLES

Certain aspects of the instant disclosure may be further understood in light of the following examples, which should not be construed as limiting. Modifications to materials and methods will be apparent to those skilled in the art.

### Example 1

### Deletion of the yitMOP Operon in Bacillus Cells

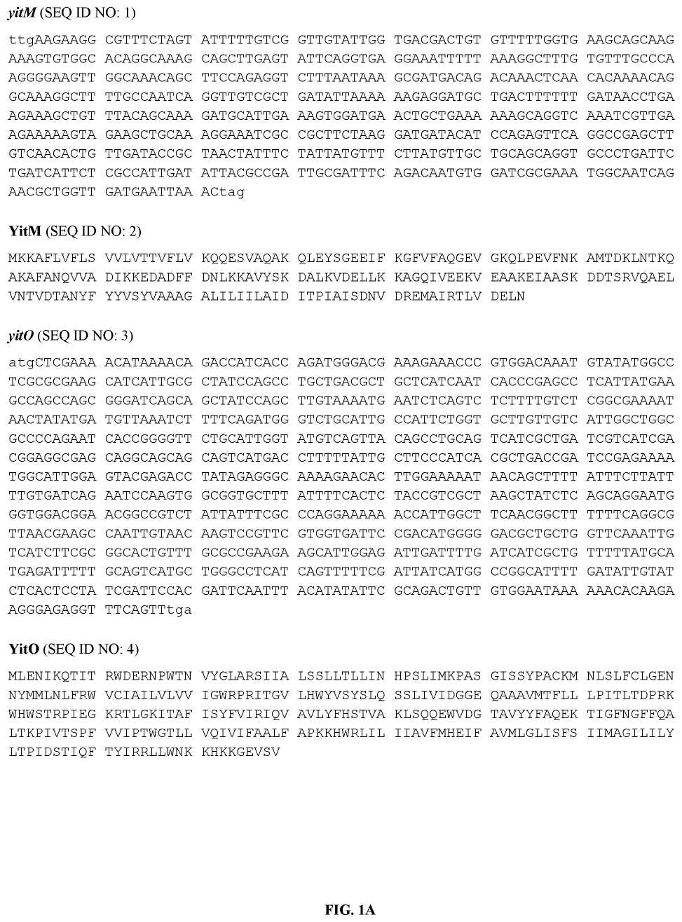

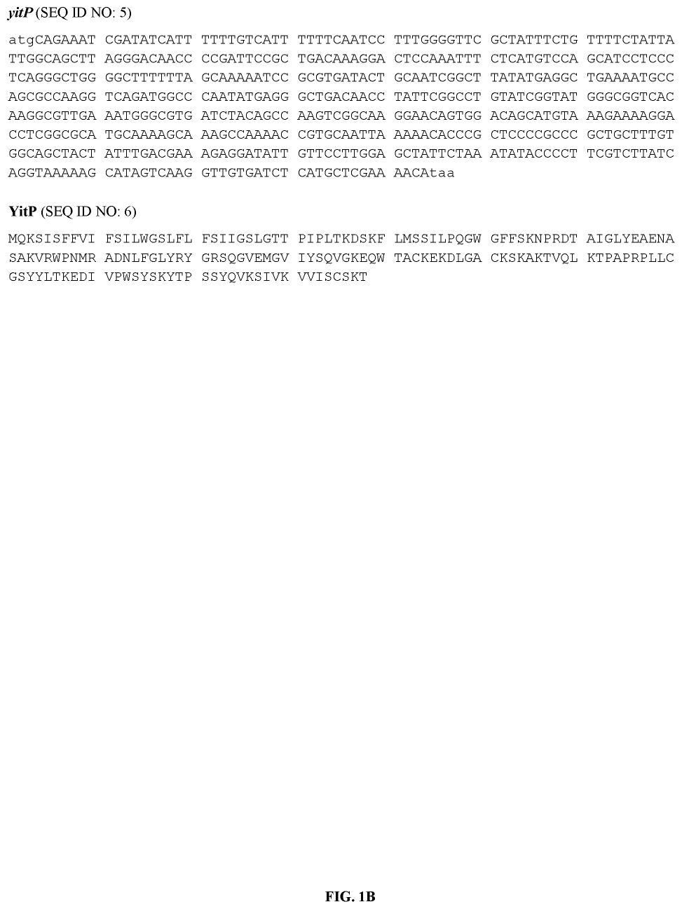

As generally set forth and described above, a novel B. subtilis cannibalism operon named yitMOP was identified as a highly expressed transcript via a transcriptome analysis of a B. subtilis strain expressing FNA protease. More particularly, as presented in FIG. 1A and FIG. 1B, the B. subtilis yitMOP operon encodes a YitM protein (SEQ ID NO: 2), a YitO protein (SEQ ID NO: 4) and a YitP protein (SEQ ID NO: 6). The instant example describes the deletion of the yitMOP operon in a parental B. subtilis cell. For example, to generate a modified B. subtilis (daughter) cell, a plasmid named “pUCyitMOP:spec” was constructed to delete of the yitMOP operon in B. subtilis. For example, the pUCyitMOP:spec plasmid was built to replace the entire yitMOP operon in B. subtilis with a spectinomycin resistance (specR) marker gene flanked by loxP sites (herein, the “loxP-specR-loxP cassette”). Thus, the upstream (5′) sequences were amplified from B. subtilis (strain 168) genomic DNA (gDNA) using the oJKL228 (SEQ ID NO: 7) and oJKL229 (SEQ ID NO: 8) primers set forth below in TABLE 1.

TABLE 1
OLIGONUCLEOTIDE PRIMERS

| | | |
|---|---|---|
| oJKL228 | CTCGGTACGACACCTGCAAACAT | SEQ ID NO: 7 |
| oJKL229 | ACCGTCGATCGACTGCTCCTTTTAAC | SEQ ID NO: 8 |

The downstream (3′) homology sequence was amplified from B. subtilis (strain 168) gDNA using the oJKL232 (SEQ ID NO: 9) and oJKL233 (SEQ ID NO: 10) primers set forth below in TABLE 2.

TABLE 2
OLIGONUCLEOTIDE PRIMERS

| | | |
|---|---|---|
| oJKL232 | CCTAGGCACATAAAAAAACGGCTC | SEQ ID NO: 9 |
| | GCTCCGC | |
| oJKL233 | TGCATGCCGAACAGGAAGGACGAT | SEQ ID NO: 10 |

The loxP-specR-loxP cassette was amplified from the Topo-specR vector using the oJKL230 (SEQ ID NO: 11) and oJKL231 (SEQ ID NO: 12) set forth below in TABLE 3. The Topo-specR vector comprises a spectinomycin adenyltransferase gene (specR) with native promoter and terminator elements, which gene was cloned into the Invitrogen Topo vector blunt and used to transform E. coli TOP10 cells.

TABLE 3
OLIGONUCLEOTIDE PRIMERS

| | | |
|---|---|---|
| oJKL230 | GCAGTCGATCGACGGTATCG | SEQ ID NO: 11 |
| | ATAAGCTG | |
| oJKL231 | TTTTATGTGCCTAGGATGCA | SEQ ID NO: 12 |
| | TATGGCG | |

The pUC vector backbone was amplified from the pUCL fragment obtained from the GeneArt Seamless Cloning kit according to manufacturer's instructions, using the oJKL226 (SEQ ID NO: 13) and oJKL227 (SEQ ID NO: 14) primers set forth below in TABLE 4.

TABLE 4
OLIGONUCLEOTIDE PRIMERS

| | | |
|---|---|---|
| oJKL226 | TCCTGTTCGGCATGCAAGCTT | SEQ ID NO: 13 |
| | GGCGTA | |
| oJKL227 | CAGGTGTCGTACCGAGCTCGA | SEQ ID NO: 14 |
| | ATTCAC | |

The vector fragments were assembled using a Gibson cloning kit and used to transform E. coli. The upstream (5′) and downstream (3′) homology sections of the vector were sequence verified using the forward (M13F; SEQ ID NO: 15) and reverse (M13R; SEQ ID NO: 16) primers set forth below in TABLE 5.

TABLE 5
OLIGONUCLEOTIDE PRIMERS

| | | |
|---|---|---|
| M13F | AGGCGATTAAGTTGGGT | SEQ ID NO: 15 |
| M13R | TCACACAGGAAACAGCTATGA | SEQ ID NO: 16 |

The sequence confirmed vector was isolated and linearized using AatII, wherein the linearized vector was then used to transform B. subtilis cells. The deletion was then verified using PCR, wherein the upstream (5′) integration junction was confirmed using the oJKL246 (SEQ ID NO: 17) and 5′ spec out (SEQ ID NO: 18) primers set forth below in TABLE 6.

TABLE 6
OLIGONUCLEOTIDE PRIMERS

| | | |
|---|---|---|
| oJKL246 | GATAAACCGCACCGACTCG | SEQ ID NO: 17 |
| 5′ spec out | AGTCCACTCTCAACTCCTGATCC | SEQ ID NO: 18 |

The downstream integration junction was confirmed using the oJKL247 (SEQ ID NO: 19) and 3′ spec out (SEQ ID NO: 20) primers set forth below in TABLE 7.

TABLE 7
OLIGONUCLEOTIDE PRIMERS

| | | |
|---|---|---|
| oJKL247 | CAGCACATCTTTCAGCTGGC | SEQ ID NO: 19 |
| 3′ spec out | AAATTGAAAAAATGGTGGAA | SEQ ID NO: 20 |
| | ACAC | |

### Example 2

### Deletion of the sdpABC Operon

The instant example describes the construction of a plasmid named “pUCsdp::tet” and its introduction into B. subtilis cells. More specifically, the pUCsdp::tet plasmid was constructed comprising upstream (5′) and downstream (3′) DNA sequences to replace the entire sdpABC operon in B. subtilis cells with a tetracycline (tetR) marker flanked by loxP sites (herein, the “loxP-tetR-loxP cassette”), wherein removal of the tetracycline marker results in a deletion of the sdpABC operon.

Thus, the upstream (5′) sdpABC sequence was amplified from B. subtilis gDNA using the oJKL220 (SEQ ID NO: 21) and oJKL221 (SEQ ID NO: 22) primers set forth below in TABLE 8.

TABLE 8
OLIGONUCLEOTIDE PRIMERS

| | | |
|---|---|---|
| oJKL220 | CTCGGTACCCCGCATTGACGTGT | SEQ ID NO: 21 |
| oJKL221 | TATCAGGATACAGTAATAATTCC | SEQ ID NO: 22 |
| | CTTTTTTTTTGATG | |

The downstream (3′) sequence was amplified from B. subtilis gDNA using the oJKL224 (SEQ ID NO: 23) and oJKL225 (SEQ ID NO: 24) primers set forth below in TABLE 9.

TABLE 9
OLIGONUCLEOTIDE PRIMERS

| | | |
|---|---|---|
| oJKL224 | CTGGAGGATCCATTATAATTGA | SEQ ID NO: 23 |
| | GTGTCTTGC | |
| oJKL225 | TGCATGCCGGATATGACTGCGG | SEQ ID NO: 24 |
| | GAGAT | |

The loxP-tetR-loxP cassette was amplified from plasmid pUCLlacA-ganAB::tet using the oJKL222 (SEQ ID NO: 25) and oJKL223 (SEQ ID NO: 26) primers set forth below in TABLE 10.

TABLE 10
OLIGONUCLEOTIDE PRIMERS

| | | |
|---|---|---|
| oJKL222 | TTACTGTATCCTGATACCGGGAG | SEQ ID NO: 25 |
| oJKL223 | ATAATGGATCCTCCAGAATGTCAGA | SEQ ID NO: 26 |

The pUC19 vector backbone was amplified from the GeneArt Linear pUC19L vector from Life Technologies according to manufacturer's instructions using the oJKL218 (SEQ ID NO: 27) and oJKL219 (SEQ ID NO: 28) primers set forth below in TABLE 11.

TABLE 11
OLIGONUCLEOTIDE PRIMERS

| | | |
|---|---|---|
| oJKL218 | TCATATCCGGCATGCAAGCTTGG | SEQ ID NO: 27 |
| oJKL219 | AATGCGGGGTACCGAGCTCGAATT | SEQ ID NO: 28 |

The upstream, downstream, loxP-tetR-loxP and pUC19 amplicons were fused together using the NEB Gibson kit following manufacturer's instructions. A sequenced verified plasmid was then linearized using AatII and used to transform B. subtilis cells. The genomic sdp operon deletion (Δsdp:loxP-tetR-loxP) was confirmed using cPCR, wherein the upstream integration site was checked using the oJKL 238 (SEQ ID NO: 29) and tet 3′ out (SEQ ID NO: 30) primers set forth below in TABLE 12.

TABLE 12
OLIGONUCLEOTIDE PRIMERS

| | | |
|---|---|---|
| oJKL238 | CAGACAATTGAATGCTTCCC | SEQ ID NO: 29 |
| tet 3′ out | TCATTGTCATTAGTTGGCTG | SEQ ID NO: 30 |
| | GTTACC | |

The downstream site was checked using the oJKL 239 (SEQ ID NO: 31) and tet 5′ out (SEQ ID NO: 32) primers set forth below in TABLE 13.

TABLE 13
OLIGONUCLEOTIDE PRIMERS

| | | |
|---|---|---|
| oJKL239 | CAAAGCTATATCAGATCCAACA | SEQ ID NO: 31 |
| tet 5′ out | ACGCTTCCCTCTTTTAATTGAA | SEQ ID NO: 32 |
| | CCC | |

Set forth below in TABLE 14 are the strain names and brief descriptions of B. subtilis cells (strains) constructed herein, and further described below in Example 3.

TABLE 14
STRAIN DESCRIPTIONS

| Strain Name | Strain Description |
|---|---|
| ZM207 | B. subtills (control) strain expressing ME-3 protease |
| ZM334 | ZM207 (daughter) strain comprising ΔsdpABC |
| ZM336 | ZM207 (daughter) strain comprising ΔyitMOP |
| ZM268 | B. subtills modified strain (ME-3 protease + ΔsdpABC + ΔyitMOP) |
| ZM434 | ZM207 (daughter) strain comprising ΔyitM |

### Example 3

### Heterologous Protease Production in Modified B. Subtilis Cells

The present example describes the production of a heterologous protein of interest (POI) in B. subtilis cells. More particularly, the parental (ZM207) and modified B. subtilis strains constructed and described above in Examples 1 and 2 (e.g., see TABLE 14), where fermented in shake flasks under identical conditions in Grant's II medium at 42° C. for forty (40) hours. The B. subtilis daughter strain named ZM434 (ΔyitM) was constructed by transforming the genomic DNA of the strain BKE11040 (trpC2 yitM::erm) obtained from Bacillus Genetic Stock Center into the parental strain (ZM207).

Subsequently, fermentation supernatants were taken and ME-3 protease activity was determined using the AAPF-assay (e.g., see PCT Publication No. WO2018/118815, incorporated herein by reference in its entirety) and monitored by the change of absorbance at 405 nm. The slope of the absorbance change (mOD/min) was reflective of the ME-3 protease activity and is reported herein for comparison. More particularly, TABLE 15 below presents the average ME-3 titers and standard errors of multiple (n≥3) biological replicates.

TABLE 15
ME-3 PROTEASE TITERS

| Strain Name | Description | mOD/minute |
|---|---|---|
| ZM207 | B. subtills (control) strain expressing ME-3 protease | 46.2 ± 1.7 |
| ZM334 | ZM207 (daughter) strain ΔsdpABC | 68.7 ± 0.3 |
| ZM336 | ZM207 (daughter) strain ΔyitMOP | 68.4 ± 0.3 |
| ZM268 | B. subtills (modified) ME-3 protease + ΔsdpABC + ΔyitMOP | 74.2 ± 0.8 |
| ZM434 | ZM207 (daughter) strain ΔyitM | 78.7 ± 0.3 |

For example, as shown in TABLE 15, both the ZM334 (ΔsdpABC) daughter strain and the ZM336 (ΔyitMOP) daughter strain produced an increased amount of the protease relative to the ZM207 parental strain. Similarly, the modified ZM268 strain (TABLE 15), comprising a combined deletion of the yitMOP and sdpABC operons (i.e., ΔyitMOP+ΔsdpABC) produces an increased amount of the ME-3 protease relative to the ZM207 (control) strain, the ZM334 strain (ΔsdpABC) and the ZM336 strain (ΔyitMOP).

In addition, it was surprisingly observed (TABLE 15) that disruption or deletion of the yitM ORF alone was sufficient to elicit the beneficial effect of increased protein production, as the ZM434 (ΔyitM) daughter strain produced slightly more ME-3 protease than the ZM336 (ΔyitMOP) daughter strain.

### Example 4

### Heterologous Protease Production in Modified B. subtilis Cells

The instant example describes the production of a heterologous protease (V42) in parental and modified B. subtilis cells under large scale fermentation conditions. More particularly, as described herein, a B. subtilis strain named CB24-12 comprises an introduced expression cassette comprising a promoter sequence “P4” positioned upstream (5′) and operably linked to an open reading frame (ORF) encoding a V42 protease which is upstream (5′) and operably linked to an ORF encoding an alanine racemase gene (alrA), wherein the cassette is integrated into the B. subtilis aprE locus (aprE::P4-V42-alrA). Likewise, a B. subtilis strain named “JL77” comprises an introduced expression cassette encoding a V42 protease (aprE::P4-V42-alrA) and a combined deletion of the yitMOP operon and the sdpABC operon (ΔyitMOP+ΔsdpABC).

Thus, as set forth in FIG. 2, the cells of the JL77 strain and CB24-12 strain were tested in large scale fermentors under standard conditions, wherein each strain was run in duplicate, and the average improvement in V42 protease production at the end of fermentation (EOF) is shown. For example, as presented in FIG. 2, the average increase in V42 protease production at the end of fermentation for the JL77 strain was approximately 10%, relative to the amount of V42 protease produced by the CB24-12 strain at the end of fermentation.

### REFERENCES

PCT Publication No. WO 2006/037483
PCT Publication No. WO1990/11352
PCT Publication No. WO1994/18314
PCT Publication No. WO1999/19467,
PCT Publication No. WO1999/43794
PCT Publication No. WO2000/29560
PCT Publication No. WO2000/60059
PCT Publication No. WO2001/51643
PCT Publication No. WO2002/14490
PCT Publication No. WO2003/083125
PCT Publication No. WO2003/089604
PCT Publication No. WO2006/037484
PCT Publication No. WO2006/089107
PCT Publication No. WO2008/112459
PCT Publication No. WO2011/072099
PCT Publication No. WO2014/164777
PCT Publication No. WO2018/118815
Albertini and Galizzi, Bacteriol., 162:1203-1211, 1985.
Botstein and Shortie, Science 229: 4719, 1985.
Brode et al., “Subtilisin BPN′ variants: increased hydrolytic activity on surface-bound substrates via decreased surface activity”, Biochemistry, 35(10):3162-3169, 1996.
Caspers et al., “Improvement of Sec-dependent secretion of a heterologous model protein in Bacillus subtilis by saturation mutagenesis of the N-domain of the AmyE signal peptide”, Appl. Microbiol. Biotechnol., 86(6):1877-1885, 2010.
Chang et al., Mol. Gen. Genet., 168:11-115, 1979.
Earl et al., “Ecology and genomics of Bacillus subtilis”, Trends in Microbiology., 16(6):269-275, 2008.
Ellermeier et al., “A Three Protein Signaling Pathway Governing Immunity to a Bacterial Cannibalism Toxin”, Cell, 134: 549-559, 2006.
Ferrari et al., “Genetics,” in Harwood et al. (ed.), Bacillus, Plenum Publishing Corp., 1989.
Fisher et. al., Arch. Microbiol., 139:213-217, 1981.
Gonzalez-Pastor et al., “Cannibalism by Sporulating Bacteria”, Science, 301: 510-513, 2003.
Guerot-Fleury, Gene, 167:335-337, 1995.
Higuchi et al., Nucleic Acids Research 16: 7351, 1988.
Ho et al., Gene 77: 61, 1989.
Hoch et al., J. Bacteriol., 93:1925-1937, 1967.
Holubova, Folia Microbiol., 30:97, 1985.
Hopwood, The Isolation of Mutants in Methods in Microbiology (J. R. Norris and D. W. Ribbons, eds.) pp 363-433, Academic Press, New York, 1970.
Horton et al., Gene 77: 61, 1989.
Iglesias and Trautner, Molecular General Genetics 189: 73-76, 1983.
Jensen et al., “Cell-associated degradation affects the yield of secreted engineered and heterologous proteins in the Bacillus subtilis expression system” Microbiology, 146 (Pt 10:2583-2594, 2000.
Lo et al., Proceedings of the National Academy of Sciences USA 81: 2285, 1985.
Mann et al., Current Microbiol., 13:131-135, 1986.
McDonald, J. Gen. Microbiol., 130:203, 1984.
Needleman and Wunsch, J. Mol. Biol., 48: 443, 1970.
Olempska-Beer et al., “Food-processing enzymes from recombinant microorganisms—a review” Regul. Toxicol. Pharmacol., 45(2): 144-158, 2006.
Palmeros et al., Gene 247:255-264, 2000.
Parish and Stoker, FEMS Microbiology Letters 154: 151-157, 1997.
Pearson and Lipman, Proc. Natl. Acad. Sci. USA 85: 2444, 1988.
Perego, 1993, In A. L. Sonneshein, J. A. Hoch, and R. Losick, editors, Bacillus subtilis and Other Gram-Positive Bacteria, Chapter 42, American Society of Microbiology, Washington, D.C.
Perez Morales et al., “Production of the Cannibalism Toxin SDP is a Multistep Process that Requires SdpA and SdpB”, J. Bacteriology, 195(14): 3244-3251, 2013.
Raul et al., “Production and partial purification of alpha amylase from Bacillus subtilis (MTCC 121) using solid state fermentation”, Biochemistry Research International, 2014.
Sarkar and Sommer, BioTechniques 8: 404, 1990.
Sauer and Henderson, “Site-specific DNA recombination in mammalian cells by the Cre recombinase of bacteriophage P1”, PNAS, 85(14): 5166-5170, 1988.
Sauer, “Functional expression of the cre-lox site-specific recombination system in the yeast Saccharomyces cerevisiae”, Mol. Cell Biol., 7(6): 2087-2096, 1987.
Saunders et al., J. Bacteriol., 157: 718-726, 1984.
Shimada, Meth. Mol. Biol. 57: 157; 1996
Silver, “Novel inhibitors of bacterial cell wall synthesis”, Curr. Opin. Microbiol. 6:431-438, 2003.
Silver, “Does the cell wall of bacteria remain a viable source of targets for novel antibiotics?”, Biochem. Pharmacol. 71:996-1005, 2006.
Smith and Waterman, Adv. Appl. Math., 2: 482, 1981.
Smith et al., Appl. Env. Microbiol., 51:634 1986.
Stahl and Ferrari, J. Bacteriol., 158:411-418, 1984.
Stahl et al, J. Bacteriol., 158:411-418, 1984.
Trieu-Cuot et al., Gene, 23:331-341, 1983.
Van Dijl and Hecker, “Bacillus subtilis: from soil bacterium to super-secreting cell factory”, Microbial Cell Factories, 12(3). 2013.
Vorobjeva et al., FEMS Microbiol. Lett., 7:261-263, 1980.
Wang et al., “Deleting multiple lytic genes enhances biomass yield and production of recombinant proteins by Bacillus subtilis”, Microbial Cell Factories 13(129): 1-11, 2014.
Westers et al., “Bacillus subtilis as cell factory for pharmaceutical proteins: a biotechnological approach to optimize the host organism”, Biochimica et Biophysica Acta., 1694:299-310, 2004.
Youngman et al., Proc. Natl. Acad. Sci. USA 80: 2305-2309, 1983.

1. A method for producing an increased amount of a protein of interest (POI) in a modified Bacillus sp. cell comprising:
(a) obtaining a parental Bacillus sp. cell producing a POI,
(b) genetically modifying the parental cell by disrupting or deleting the yitMOP operon, and
(c) fermenting the modified cell under suitable conditions for the production of the POI,
wherein the modified cell produces an increased amount of the POI relative to the parental cell when fermented under the same conditions.

2. A method for producing an increased amount of a protein of interest (POI) in a modified Bacillus sp. cell comprising:
(a) obtaining a parental Bacillus sp. cell producing a POI,
(b) genetically modifying the parental Bacillus sp. cell by disrupting or deleting a regulatory region of the yitMOP operon, thereby rendering the modified cell deficient in the production of functional YitM, YitO and/or YitP proteins, and
(c) fermenting the modified cell under suitable conditions for the production of the POI,
wherein the modified cell produces an increased amount of the POI relative to the parental cell when fermented under the same conditions.

3. A method for producing an increased amount of a protein of interest (POI) in a modified Bacillus sp. cell comprising:
(a) obtaining a parental Bacillus sp. cell producing a POI,
(b) genetically modifying the parental cell by disrupting or deleting the yitM gene, and
(c) fermenting the modified cell under suitable conditions for the production of the POI,
wherein the modified cell produces an increased amount of the POI relative to the parental cell when fermented under the same conditions.

4. (canceled)

5. The method of claim 1, wherein the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus and B. thuringiensis.

6. The method of claim 1, wherein the POI is an enzyme.

7. The method of claim 6, wherein the enzyme is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.

8. A genetically modified Bacillus sp. cell produced according to the method of claim 1.

9. A genetically modified Bacillus sp. cell derived from a parental Bacillus sp. cell producing a protein of interest (POI), wherein the modified cell comprises a disrupted or deleted yitMOP operon, and produces an increased amount of the POI relative to the parental cell when fermented under the same conditions.

10-15. (canceled)

16. The method of claim 2, wherein the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus and B. thuringiensis.

17. The method of claim 2, wherein the POI is an enzyme.

18. The method of claim 17, wherein the enzyme is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.

19. The method of claim 3, wherein the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus and B. thuringiensis.

20. The method of claim 3, wherein the POI is an enzyme.

21. The method of claim 20, wherein the enzyme is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.


SEQUENCE LISTING (47 sequences)

SEQ ID NO: 1 - DNA, 585 bp, Bacillus subtilis
ttgaagaaggcgtttctagtatttttgtcggttgtattggtgacgactgtgtttttggtgaagcagcaagaaagtgtggcacaggcaaagcagcttgagtattcaggtgaggaaatttttaaaggctttgtgtttgcccaaggggaagttggcaaacagcttccagaggtctttaataaagcgatgacagacaaactcaacacaaaacaggcaaaggcttttgccaatcaggttgtcgctgatattaaaaaagaggatgctgacttttttgataacctgaagaaagctgtttacagcaaagatgcattgaaagtggatgaactgctgaaaaaagcaggtcaaatcgttgaagaaaaagtagaagctgcaaaggaaatcgccgcttctaaggatgatacatccagagttcaggccgagcttgtcaacactgttgataccgctaactatttctattatgtttcttatgttgctgcagcaggtgccctgattctgatcattctcgccattgatattacgccgattgcgatttcagacaatgtggatcgcgaaatggcaatcagaacgctggttgatgaattaaactag

SEQ ID NO: 2 - protein, 194 aa, Bacillus subtilis
MKKAFLVFLSVVLVTTVFLVKQQESVAQAKQLEYSGEEIFKGFVFAQGEVGKQLPEVFNKAMTDKLNTKQAKAFANQVVADIKKEDADFFDNLKKAVYSKDALKVDELLKKAGQIVEEKVEAAKEIAASKDDTSRVQAELVNTVDTANYFYYVSYVAAAGALILIILAIDITPIAISDNVDREMAIRTLVDELN

SEQ ID NO: 3 - DNA, 930 bp, Bacillus subtilis
atgctcgaaaacataaaacagaccatcaccagatgggacgaaagaaacccgtggacaaatgtatatggcctcgcgcgaagcatcattgcgctatccagcctgctgacgctgctcatcaatcacccgagcctcattatgaagccagccagcgggatcagcagctatccagcttgtaaaatgaatctcagtctcttttgtctcggcgaaaataactatatgatgttaaatcttttcagatgggtctgcattgccattctggtgcttgttgtcattggctggcgccccagaatcaccggggttctgcattggtatgtcagttacagcctgcagtcatcgctgatcgtcatcgacggaggcgagcaggcagcagcagtcatgacctttttattgcttcccatcacgctgaccgatccgagaaaatggcattggagtacgagacctatagagggcaaaagaacacttggaaaaataacagcttttatttcttattttgtgatcagaatccaagtggcggtgctttattttcactctaccgtcgctaagctatctcagcaggaatgggtggacggaacggccgtctattatttcgcccaggaaaaaaccattggcttcaacggcttttttcaggcgttaacgaagccaattgtaacaagtccgttcgtggtgattccgacatgggggacgctgctggttcaaattgtcatcttcgcggcactgtttgcgccgaagaagcattggagattgattttgatcatcgctgtttttatgcatgagatttttgcagtcatgctgggcctcatcagtttttcgattatcatggccggcattttgatattgtatctcactcctatcgattccacgattcaatttacatatattcgcagactgttgtggaataaaaaacacaagaagggagaggtttcagtttga

SEQ ID NO: 4 - protein, 309 aa, Bacillus subtilis
MLENIKQTITRWDERNPWTNVYGLARSIIALSSLLTLLINHPSLIMKPASGISSYPACKMNLSLFCLGENNYMMLNLFRWVCIAILVLVVIGWRPRITGVLHWYVSYSLQSSLIVIDGGEQAAAVMTFLLLPITLTDPRKWHWSTRPIEGKRTLGKITAFISYFVIRIQVAVLYFHSTVAKLSQQEWVDGTAVYYFAQEKTIGFNGFFQALTKPIVTSPFVVIPTWGTLLVQIVIFAALFAPKKHWRLILIIAVFMHEIFAVMLGLISFSIIMAGILILYLTPIDSTIQFTYIRRLLWNKKHKKGEVSV

SEQ ID NO: 5 - DNA, 537 bp, Bacillus subtilis
atgcagaaatcgatatcattttttgtcattttttcaatcctttggggttcgctatttctgttttctattattggcagcttagggacaaccccgattccgctgacaaaggactccaaatttctcatgtccagcatcctccctcagggctggggcttttttagcaaaaatccgcgtgatactgcaatcggcttatatgaggctgaaaatgccagcgccaaggtcagatggcccaatatgagggctgacaacctattcggcctgtatcggtatgggcggtcacaaggcgttgaaatgggcgtgatctacagccaagtcggcaaggaacagtggacagcatgtaaagaaaaggacctcggcgcatgcaaaagcaaagccaaaaccgtgcaattaaaaacacccgctccccgcccgctgctttgtggcagctactatttgacgaaagaggatattgttccttggagctattctaaatataccccttcgtcttatcaggtaaaaagcatagtcaaggttgtgatctcatgctcgaaaacataa

SEQ ID NO: 6 - protein, 178 aa, Bacillus subtilis
MQKSISFFVIFSILWGSLFLFSIIGSLGTTPIPLTKDSKFLMSSILPQGWGFFSKNPRDTAIGLYEAENASAKVRWPNMRADNLFGLYRYGRSQGVEMGVIYSQVGKEQWTACKEKDLGACKSKAKTVQLKTPAPRPLLCGSYYLTKEDIVPWSYSKYTPSSYQVKSIVKVVISCSKT

SEQ ID NO: 7 - DNA, 23 bp, Artificial Sequence
  primer
ctcggtacgacacctgcaaacat

SEQ ID NO: 8 - DNA, 26 bp, Artificial Sequence
  primer
accgtcgatcgactgctccttttaac

SEQ ID NO: 9 - DNA, 31 bp, Artificial Sequence
  primer
cctaggcacataaaaaaacggctcgctccgc

SEQ ID NO: 10 - DNA, 24 bp, Artificial Sequence
  primer
tgcatgccgaacaggaaggacgat

SEQ ID NO: 11 - DNA, 28 bp, Artificial Sequence
  primer
gcagtcgatcgacggtatcgataagctg

SEQ ID NO: 12 - DNA, 27 bp, Artificial Sequence
  primer
ttttatgtgcctaggatgcatatggcg

SEQ ID NO: 13 - DNA, 27 bp, Artificial Sequence
  primer
tcctgttcggcatgcaagcttggcgta

SEQ ID NO: 14 - DNA, 27 bp, Artificial Sequence
  primer
caggtgtcgtaccgagctcgaattcac

SEQ ID NO: 15 - DNA, 17 bp, Artificial Sequence
  primer
aggcgattaagttgggt

SEQ ID NO: 16 - DNA, 21 bp, Artificial Sequence
  primer
tcacacaggaaacagctatga

SEQ ID NO: 17 - DNA, 19 bp, Artificial Sequence
  primer
gataaaccgcaccgactcg

SEQ ID NO: 18 - DNA, 23 bp, Artificial Sequence
  primer
agtccactctcaactcctgatcc

SEQ ID NO: 19 - DNA, 20 bp, Artificial Sequence
  primer
cagcacatctttcagctggc

SEQ ID NO: 20 - DNA, 24 bp, Artificial Sequence
  primer
aaattgaaaaaatggtggaaacac

SEQ ID NO: 21 - DNA, 23 bp, Artificial Sequence
  primer
ctcggtaccccgcattgacgtgt

SEQ ID NO: 22 - DNA, 37 bp, Artificial Sequence
  primer
tatcaggatacagtaataattccctttttttttgatg

SEQ ID NO: 23 - DNA, 31 bp, Artificial Sequence
  primer
ctggaggatccattataattgagtgtcttgc

SEQ ID NO: 24 - DNA, 27 bp, Artificial Sequence
  primer
tgcatgccggatatgactgcgggagat

SEQ ID NO: 25 - DNA, 23 bp, Artificial Sequence
  primer
ttactgtatcctgataccgggag

SEQ ID NO: 26 - DNA, 25 bp, Artificial Sequence
  primer
ataatggatcctccagaatgtcaga

SEQ ID NO: 27 - DNA, 23 bp, Artificial Sequence
  primer
tcatatccggcatgcaagcttgg

SEQ ID NO: 28 - DNA, 24 bp, Artificial Sequence
  primer
aatgcggggtaccgagctcgaatt

SEQ ID NO: 29 - DNA, 20 bp, Artificial Sequence
  primer
cagacaattgaatgcttccc

SEQ ID NO: 30 - DNA, 26 bp, Artificial Sequence
  primer
tcattgtcattagttggctggttacc

SEQ ID NO: 31 - DNA, 22 bp, Artificial Sequence
  primer
caaagctatatcagatccaaca

SEQ ID NO: 32 - DNA, 25 bp, Artificial Sequence
  primer
acgcttccctcttttaattgaaccc

SEQ ID NO: 33 - DNA, 531 bp, Bacillus amyloliquefaciens
ttgactgacaagagacaaaacagcatgttttcattctttctttttataatggccgcctggatcatcgttttgtttttaaccgtcatcgcggcactgccaagctccgccatccaattaaccaagcatcaatccaaaaccgtttccatgatttatccgcaaagctggggatttttcagtaaagatccgagagaagacagtctgaacatctatctccttgatggcgatgaaaagctcagatggccgaataattcactgaaaaatgtgatcggcttaaaccgatacgggcggacgcaagggatagagctcggccttatgctggaaaagctgccttccaaagagaagtggcacgatttcacggctggccaggactttaagaataccatatttttgaaaatgaaaaatgagtcgccctccccaaggctgaaagggaaaatgatcattacatatgaacgcattaccccatgggcatggagcaaacataccgaaacgaaacgaaccgttaaaaaatacatcggagtggatgtcaattga

SEQ ID NO: 34 - protein, 176 aa, Bacillus amyloliquefaciens
MTDKRQNSMFSFFLFIMAAWIIVLFLTVIAALPSSAIQLTKHQSKTVSMIYPQSWGFFSKDPREDSLNIYLLDGDEKLRWPNNSLKNVIGLNRYGRTQGIELGLMLEKLPSKEKWHDFTAGQDFKNTIFLKMKNESPSPRLKGKMIITYERITPWAWSKHTETKRTVKKYIGVDVN

SEQ ID NO: 35 - DNA, 945 bp, Bacillus amyloliquefaciens
ttgattcaggatgcaaaaaagcagctcactttatggatgatgcagaatccgccttggacaaatgtgtacgggcttgccagaagcttaatcgcgctcggcacgctgctgacattgcttttcaatccggcgtctgtcttcttcaaaacgcaaaatcaaatgctcagcttttccgacacgttcagtttgtttttgctgtttcccgatttccgttacattgagatcgtcaaagccgtttgtatctgtattttaattctggttgtcattggctggcggccccgcctcaccggtttttttcattggtggatttcgtacagcttccaaaactccgccgttactcttgacggaggagatcaggtggctgctgtttttacgctgctgctgcttccgctgacattgactgatccgcggaaatggcactggtctccgtacgaggcgtcttttgagaagaggccgcttgagccttactaccgttcgttatgctgggcgacgctgacgatcatcagaatacaggtcgcgatgctgtactttaactccgtcattgccaagctgatggaagaagaatggcagaacggaaccgccgtttattactatttgaacgatccgatgctggggctgccggatgcgcttcacagtctgtttgactttatcctgcagtcaaagcttgtcatcatcccgacttggggcacgctcattgtgcaaaccattatgttctgcgctttgttcgctccgaaaaaatattggcgtctgatattgtttatatccattttcatgcatgaaatctttgctgttatgctggggcttacctcgttctccattacggtagctggtattttagacctgtttttggtaccggcagagaaaacagtagctttgaaccattacaatccgctttttatcagatacaattttctcaataaaaggaaaaggaggaaattatatggttaa

SEQ ID NO: 36 - protein, 314 aa, Bacillus amyloliquefaciens
MIQDAKKQLTLWMMQNPPWTNVYGLARSLIALGTLLTLLFNPASVFFKTQNQMLSFSDTFSLFLLFPDFRYIEIVKAVCICILILVVIGWRPRLTGFFHWWISYSFQNSAVTLDGGDQVAAVFTLLLLPLTLTDPRKWHWSPYEASFEKRPLEPYYRSLCWATLTIIRIQVAMLYFNSVIAKLMEEEWQNGTAVYYYLNDPMLGLPDALHSLFDFILQSKLVIIPTWGTLIVQTIMFCALFAPKKYWRLILFISIFMHEIFAVMLGLTSFSITVAGILDLFLVPAEKTVALNHYNPLFIRYNFLNKRKRRKLYG

SEQ ID NO: 37 - DNA, 591 bp, Bacillus amyloliquefaciens
atggttaaaagcaaaaaatccgctgccgccatgatgctggtgtttgttttaatgatcgcattagtcggcccgcatgccacagcgaaaactgaggcatccgtctcttattcgggtgaagacattttttccggcatctttttcggacaaggtgaggtcgcaaagcttcttcctgaagtatgggatacgaaagccgtcaaaaagattaatcaggatgaattaaagcaagtttccgatcaggtgatcgctgatattaaaaagtcatcgccggattattttaagaagttaaaagcggcggtctacagcggcgattatgagaaagtgaacagcgtcatcgatcaaggctcacagattatggcgaaagtgctgaaagaagagtttgacaaaaaaagcatggacaaaggaaaatcaacggccatcggacaatgcgcaacggctgtatgggtatttttaaacgcggcggctgccatagagacgatcgctcttgccgtagcggccatcattttgacggtagcaagcgttgacgaagattccgacttaggaaaagagcagctcatcgcaaatgtcgttgaccgcattaatgcgcagcactga

SEQ ID NO: 38 - protein, 196 aa, Bacillus amyloliquefaciens
MVKSKKSAAAMMLVFVLMIALVGPHATAKTEASVSYSGEDIFSGIFFGQGEVAKLLPEVWDTKAVKKINQDELKQVSDQVIADIKKSSPDYFKKLKAAVYSGDYEKVNSVIDQGSQIMAKVLKEEFDKKSMDKGKSTAIGQCATAVWVFLNAAAAIETIALAVAAIILTVASVDEDSDLGKEQLIANVVDRINAQH

SEQ ID NO: 39 - DNA, 122 bp, Bacillus amyloliquefaciens
atgaacactcctttttacatataatagtatattagtccaaaaaatctaatttctcaatattttacctgttgaatggtgtataatcatgtcaatgaaatgtccattcgataggagggtttcaa

SEQ ID NO: 40 - DNA, 177 bp, Bacillus subtilis
ggtttaaatatatcgaaaaaaacactatatatcaatcacatatagaagtgtaaaatgtcctgtttttccttttaacaaaaatggaatttctcatactttttaacctttttattacataaatgtttttatattatgaaaagtgtaaaaacgattcttcaggttaaaaggagcagtcga

SEQ ID NO: 41 - DNA, 221 bp, Bacillus subtilis
aaagttcccaaattcaattctgggaacttttttaagtccaatccaaatggttgaatatcaaacttcaagaaaacaaacaaaataatgcataatttacattaatttattaattatccattttttgttgattattctgactagctattatataatctttttgaaatgattatattagcttagaggaggtaatctacatcaaaaaaaagggaattattactgta

SEQ ID NO: 42 - DNA, 477 bp, Bacillus subtilis
atgactatatgtttcctattattttcttcttattactttagcaatatttcacctcagaatccactgttcaaaaaaaattttttgcaacaattgtctccccaaggctttggcttttatagtaaaagccctacagaagaaaacatttcatttcacacaaaagaaaatttaaagttacctaatgcacttcccaataatttttttgggataaaaagagaaggaagagttcaggcaatagaattaggcaaaattgtagagaatatcgatccaaagaattggaaaacttgtgaaaacaacaactcctgcacaaatttagagaaacaaataaagcctattaaggttataaaaaatgaagattatatacatcttagcaaaggagaatacctaatatatcgccaaaaaccactctcatggtattggatagactttaagcaaactacctcttttgaaagaaaggtgctaaaaataaaaatagtatga

SEQ ID NO: 43 - protein, 158 aa, Bacillus subtilis
MTICFLLFSSYYFSNISPQNPLFKKNFLQQLSPQGFGFYSKSPTEENISFHTKENLKLPNALPNNFFGIKREGRVQAIELGKIVENIDPKNWKTCENNNSCTNLEKQIKPIKVIKNEDYIHLSKGEYLIYRQKPLSWYWIDFKQTTSFERKVLKIKIV

SEQ ID NO: 44 - DNA, 972 bp, Bacillus subtilis
atgaagatattaaatagtttagaaggttatattgacacctataatccatggaaaaatacatatgcactttttagaagtttacttggtttctcaacattactagtactattattcaatagtactgatattttatttagttatagtgcaaataatgtcacatgtgaaaatgtctatatccctaccgctttttgttttgctaaagaatatagtatcaattttgagattataagatacttaatgatttttatattaaccttagtggttatagggtggagacctagatttaccggtttatttcactggtatatttgctatagtattcaaacttcagctttaactatcgatggtggagagcaaattgcaactgttctttcttttcttatattacctgttacattattagattcaaggcgaaatcattggaatataaagaaaaacaataatgaatctttcacaaagaagacagtattgttttatataatgacaataattaaaattcaagtttttatcatttatttaaacgcagctttagagcgattgaaaaataaagagtgggcagaaggaacagcaatttactatttcttttctgatccggtgtttggattacctgaatatcaacttaacttaatgaatccactacttgaaagcaattttattgttgtcatcacttggttagtaactatttttgagttgttcttagcagcaagcataatttcaaatatcagaataaagagaattgcccttgttttgggaatattatttcatattgggataatattcagcattggtattgtaagttttggcttgatcatgatatcagcattaattatatatctgcatcctgtacaacaaaatatcactatgaattggtgttctcctttatttaaatatatatatgtaaaaggaaagagaaatttcaaaagaataggaggtgaatcagtcaagtttcttacaaaattgtttcatagctaa

SEQ ID NO: 45 - protein, 323 aa, Bacillus subtilis
MKILNSLEGYIDTYNPWKNTYALFRSLLGFSTLLVLLFNSTDILFSYSANNVTCENVYIPTAFCFAKEYSINFEIIRYLMIFILTLVVIGWRPRFTGLFHWYICYSIQTSALTIDGGEQIATVLSFLILPVTLLDSRRNHWNIKKNNNESFTKKTVLFYIMTIIKIQVFIIYLNAALERLKNKEWAEGTAIYYFFSDPVFGLPEYQLNLMNPLLESNFIVVITWLVTIFELFLAASIISNIRIKRIALVLGILFHIGIIFSIGIVSFGLIMISALIIYLHPVQQNITMNWCSPLFKYIYVKGKRNFKRIGGESVKFLTKLFHS

SEQ ID NO: 46 - DNA, 612 bp, Bacillus subtilis
ttgaaaagtaaattacttaggctattgattgtttccatggtaacgatattggttttttcattagtaggactctctaaggagtcaagtacatctgctaaagaaaaccatacattttctggagaagattactttagaggacttttatttggacaaggggaagttggtaaattaatttcaaacgatttggaccctaaactcgtaaaagaggcaaatagtacagaaggtaaaaagttagtaaatgatgtagtcaaatttataaaaaaagatcaaccacaatatatggatgaattgaaacaatcgattgacagcaaagaccctaaaaaactcattgaaaatatgaccaaagcagaccaacttatccaaaaatatgctaagaaaaatgaaaacgtaaaatactcttctaataaagttactccatcttgtgggctttatgccgtctgtgtagcagctggatatttatatgttgtgggcgttaacgcagttgcattacaaacggctgccgcagtaacaactgcagtgtggaaatacgttgccaaatattcctcttcagcttctaataattctgatttagaagcggctgctgcaaaaaccctaaaattgattcatcaataa

SEQ ID NO: 47 - protein, 203 aa, Bacillus subtilis
MKSKLLRLLIVSMVTILVFSLVGLSKESSTSAKENHTFSGEDYFRGLLFGQGEVGKLISNDLDPKLVKEANSTEGKKLVNDVVKFIKKDQPQYMDELKQSIDSKDPKKLIENMTKADQLIQKYAKKNENVKYSSNKVTPSCGLYAVCVAAGYLYVVGVNAVALQTAAAVTTAVWKYVAKYSSSASNNSDLEAAAAKTLKLIHQ